(19) United States
(12) Patent Application Publication
(10) Pub. No.: US 2022/0325079 A1
(43) Pub. Date: Oct. 13, 2022

(54) COMPOSTABLE AIDS FOR EATING OR DRINKING MADE OF VEGETABLE STARCH AND VEGETABLE THICKENER OR GELLING AGENT AND METHODS FOR PRODUCING SAME

(71) Applicant: HOPE TREE INTERNATIONAL GMBH, HOLZKIRCHEN (DE)

(72) Inventor: LAMBERT DUSTIN DINZINGER, GERETSRIED-GELTING (DE)

(21) Appl. No.: 17/634,973

(22) PCT Filed: Aug. 13, 2020
(86) PCT No.: PCT/EP2020/072820

§ 371 Date: Feb. 12, 2022

(30) Foreign Application Priority Data
Aug. 13, 2019 (DE) 10 2019 212 126.8
May 11, 2020 (EP) 20173968.7

(51) Int. Cl. C08L 3/02

(52) U.S. Cl. CPC C08L 3/02; A47G 21/18

(57) ABSTRACT

The present invention relates to compostable tools for eating and for drinking, such as drinking straws or cutlery. The present invention furthermore relates to biodegradable medical commodities such as cotton swabs, tongue depressors or disposable toothbrushes. The tools and the medical commodities of the present invention comprise plant starch and plant-based thickener and gelling agents and can furthermore comprise cellulose/mechanical pulp, wax, oil and/or glycerine. The tools and the medical commodities of the present invention can furthermore comprise an outer coating which preferably comprises wax and/or a bipolymer, preferably rubber. The present invention additionally relates to a method for producing compostable tools for eating or drinking and for biodegradable medical commodities.

### TECHNICAL FIELD OF THE INVENTION

The present invention relates to biodegradable instruments or aids for eating or drinking comprising drinking straws/straws and cutlery and methods for producing same. Using vegetable starch and a vegetable thickener or a gelling agent, recyclable and biodegradable aids for eating or drinking can be produced. The addition of cellulose, wood pulp, wax, in particular carnauba wax, and/or biopolymers, in particular natural latex, can provide further advantageous properties. In this regard, aids for eating or drinking can be produced in semi-transparent or opaque embodiments. Optionally, the color of the aids can be modified by adding food coloring. The same components used for the production of the aids are also suitable for the production of medical supplies such as oral spatulas or ear swabs. The present invention thus additionally relates to biodegradable medical supplies and their methods of manufacture.

### CROSS-REFERENCE TO RELATED APPLICATIONS

This application is a national phase entry under 35 U.S.C. § 371 of International Patent Application PCT/EP2020/072820, filed Aug. 13, 2020, published as International Patent Publication WO2021/028555 on Feb. 18, 2021, which claims the benefit of German Patent Application DE 10 2019 212 126.8, filed on Aug. 13, 2019, and European Patent Application No. 20 173 968.7, filed on May 11, 2020, the contents of all are hereby incorporated by reference in their entirety.

### BACKGROUND OF THE INVENTION

In Germany alone, thousands of drinking straws and disposable cutlery are consumed each day. Conventional disposable drinking straws and disposable cutlery are neither recyclable nor compostable but can only be incinerated or recycled at enormous cost and energy, thus harming the preservation of our ecosystem. As a result, there is an enormous need for an environmentally friendly, biodegradable and compostable alternatives to not only conventional drinking straws and cutlery but also other aids for eating or drinking such as compostable lollipop handles or lollipop sticks. A biodegradable and compostable solution is also desirable for medical supplies, for example, ear swabs or cotton swabs for taking samples.

Such alternatives are provided by the aids for eating or drinking of the present invention, as well as the methods for their production. While conventional aids for eating or drinking, for instance, drinking straws and cutlery, are typically made of plastic, the aids for eating or drinking according to the present invention are made of purely vegetable materials, such as vegetable starch, a vegetable thickener agent or a gelling agent, cellulose, wood pulp and carnauba wax. Thus, the drinking straws and cutlery, for example, are not only easy to recycle or compost, but they are also produced from renewable raw materials that provide a positive carbon footprint. Therefore, the presently disclosed aids for eating or drinking constitute an environmentally friendly alternative to common solutions such as disposable plastic drinking straws.

These same materials disclosed herein can also be used for the production of medical supplies. Accordingly, the medical supplies of the present invention also form an environmentally friendly alternative to commonly used solutions such as plastic ear swabs.

### SUMMARY OF THE INVENTION

The present invention relates to instruments that aid eating and drinking in addition to medical supplies and methods for the manufacture of same.

The aids for eating and drinking according to the present invention are compostable.

The aids for eating and drinking according to the present invention are compostable within a period of 4-8 weeks, preferably compostable within 4 weeks.

The aids for eating and drinking according to the present invention are fully biodegradable within a period of 50 days.

The present invention relates to aids for eating or drinking comprising vegetable starch and vegetable thickening agents or gelling agents.

In some embodiments, the aids for eating or drinking may comprise other components. The aids for eating or drinking according to the present invention may further comprise cellulose and/or wood pulp.

The aids for eating or drinking according to the present invention may further comprise wax, preferably carnauba wax or soy wax.

The aids for eating or drinking according to the present invention may further comprise oil, preferably nut-based oils or sunflower oil.

The aids for eating or drinking according to the present invention may further comprise glycerin.

The aids for eating or drinking according to the present invention may further comprise an outer coating comprising carnauba wax, and/or canola wax, and/or kerosene, or mixtures thereof. Preferably, one or more biopolymers, particularly natural latex, have been added to the coating. A wax coating comprising carnauba wax that can additionally contain natural latex is particularly preferred.

The coating of the aids for eating or drinking according to the present invention may further comprise excipients and/or anti-stick additives.

The outer wax layer also comprises the coating of interior surfaces of the aids, such as those surfaces present in a drinking straw.

In some embodiments, the vegetable starch comprises wheat starch, potato starch, corn starch, tapioca starch, or starch from cassava, tuberous bean, batata, yam, tuberous snap pea, arakacha, tuberous wood sorrel, tuberous nasturtium, ulluco, East Indian arrowroot, arrowroot, achira, taro, tannia, white water lily, yellow pond lily, or chayote.

In some embodiments, the vegetable starch is wheat starch, potato starch, corn starch, tapioca starch, or a mixture thereof.

In some embodiments, the vegetable thickening agent or gelling agent comprises agar-agar, guar gum, xanthan gum, or a mixture of guar gum and xanthan gum.

In some embodiments, the vegetable thickening agent or gelling agent is a mixture of guar gum and xanthan gum.

In some embodiments, the aid for eating or drinking comprises
40-50% vegetable starch,
35-45% guar gum, and
1-5% xanthan gum.

In some embodiments, the aid for eating or drinking comprises
40-50% vegetable starch,
35-45% guar gum,
1-5% xanthan gum, and
a wax and/or natural latex-based coating.

The aid for eating or drinking according to the present invention may be a drinking straw, cup, cutlery, a lollipop handle/lollipop stick. The aid for drinking according to the present invention is preferably a drinking straw. The aid for eating of the present invention is preferably a lollipop handle/lollipop stick.

In one specific embodiment, the aid for eating or for drinking comprises
40-50% starch,
35-45% guar gum,
1-5% xanthan gum,
colorant, and a
natural latex-based coating,

wherein the coating consists of 5% carnauba wax, 2% glycerin, 93% natural latex, and

wherein the aid is a compostable drinking straw.

The present invention also relates to medical supplies.

The medical supplies of the present invention are compostable.

The medical supplies according to the present invention are compostable within a period of 4-8 weeks, preferably compostable within a period of 4 weeks.

The medical supplies of the present invention are biodegradable within a period of 50 days.

The present invention also relates to medical supplies comprising vegetable starch and a vegetable thickener or a gelling agent.

In some embodiments, the medical supplies may comprise other components.

The medical supplies according to the present invention may further comprise cellulose and/or wood pulp.

The medical supplies according to the present invention may further comprise wax, preferably carnauba wax or soy wax.

The medical supplies according to the present invention may further comprise oil, preferably nut oil or sunflower oil.

The medical supplies according to the present invention may further comprise glycerin.

The medical supplies according to the present invention may further comprise an outer coating comprising carnauba wax, and/or canola wax, and/or kerosene, or mixtures thereof. Preferably, the coating comprises biopolymer, particularly natural latex. Particularly preferred is a coating comprising carnauba wax, which additionally comprises natural latex.

In some embodiments, the vegetable starch comprises wheat starch, potato starch, corn starch, tapioca starch, or starch from cassava, tuber bean, batata, yam, tuber vetch, arakacha, tuberous wood sorrel, tuberous nasturtium, ulluco, East Indian arrowroot, arrowroot, achira, taro, tannia, white water lily, yellow pond lily, or chayote.

In some embodiments, the vegetable starch is wheat starch, potato starch, corn starch, tapioca starch, or a mixture thereof.

In some embodiments, the vegetable thickener or gelling agent comprises agar-agar, guar gum, xanthan gum, or a mixture of guar gum and xanthan gum.

In some embodiments, the vegetable thickener or gelling agent is a mixture of guar gum and xanthan gum.

In some embodiments, the medical supplies comprise
40-50% vegetable starch,
35-45% guar gum, and
1-5% xanthan gum.

In some embodiments, the medical supplies comprise
40-50% vegetable starch,
35-45% guar gum
1-5% xanthan gum, and
a wax-based coating.

In some embodiments, the medical supply is a cotton swab, an ear swab, a tongue depressor, or a disposable toothbrush. Preferably, the medical supply is an ear swab.

The present invention further relates to a method for the production of compostable aids for eating or drinking. In addition, the present invention relates to a method for the production of compostable medical supplies.

The present invention further relates to a method for the production of aids for eating or drinking or medical supplies, the method comprising the following steps:
a. Preparing a mixture comprising vegetable starch and a vegetable thickening or a gelling agent, comprising the steps of:
   a.1 combining the solid components of the mixture comprising vegetable starch and the vegetable thickening agent and the gelling agent and mixing same,
   a.2 adding one or more liquid components comprising water to the mixture of step a.1 in an amount of 30-60% of the net weight of the mixture of a.1 and mixing same,
b. forming the mixture from step a. into an aid for eating or drinking or into a medical supply,
c. curing the aid for eating or drinking or the medical supply formed in step b.,
d. optionally, coating the aid for eating or drinking or the medical supply cured in step c. with an emulsion comprising carnauba wax, and/or canola wax, and/or kerosene, or mixtures thereof. Particularly preferred is an emulsion comprising carnauba wax that additionally contains natural latex.

The present invention also relates to a method for the production of aids for eating or drinking or medical supplies, the method comprising the steps of:
a. Preparing a mixture comprising vegetable starch and a vegetable thickening or a gelling agent, comprising the steps of
   a.1 combining solid components of the mixture comprising:
      50-70% vegetable starch, and
      30-50% vegetable thickening and gelling agent,
      and mixing same,
   a.2 adding liquid components comprising water to the mixture of step a.1 in an amount of 30-60% of the net weight of the mixture of step a.1 and mixing same,
b. forming the mixture from step a. into an aid for eating or drinking or a medical supply,
c. curing the aid for eating or drinking or medical supply formed in step b.,
d. optionally, coating the aid for eating or drinking or medical supply cured in step c. with an emulsion comprising carnauba wax, and/or canola wax, and/or kerosene, or mixtures thereof. Particularly preferred is an emulsion comprising carnauba wax that additionally comprises natural latex.

In some embodiments, the vegetable starch in the mixture of step a.1 comprises wheat starch, potato starch, corn starch, tapioca starch, or starch from cassava, tuberous bean, batata, yam, tuberous vetch, arakacha, tuberous wood sorrel, tuberous nasturtium, ulluco, East Indian arrowroot, arrowroot, achira, taro, tannia, white water lily, yellow pond lily, or chayote.

In some embodiments, the vegetable starch of the mixture in step a.1 can be wheat starch, potato starch, corn starch, tapioca starch, or a mixture thereof.

In some embodiments, the vegetable thickener or gelling agent in the mixture of step a.1 comprises agar-agar, guar gum, xanthan gum, or a mixture of guar gum and xanthan gum.

In some embodiments, the vegetable thickening or gelling agent in the mixture of step a.1 is a mixture of guar gum and xanthan gum.

In some embodiments, the liquid components of step a.2 further comprise oil, preferably a nut oil or sunflower oil, glycerin, and/or a wax emulsion.

In a preferred embodiment, the method for the production of aids for eating or drinking or medical supplies comprises the following steps:
a. Preparing a mixture comprising vegetable starch and vegetable thickening or a gelling agent, comprising the steps of:
   a.1 combining solid components of the mixture comprising:
      50% wheat starch,
      45% guar gum,
      5% xanthan gum,
      and mixing same,
   a.2 adding liquid components to the mixture of step a.1 based on the net weight of the mixture from step a.1 comprising:
      2% carnauba wax emulsion,
      4% sunflower oil,
      5% glycerin, and
      40% water,
      and mixing same,
b. forming the mixture from step a. into an aid for eating or drinking or a medical supply,
c. curing the aid for eating or drinking or medical supply formed in step b,
d. optionally, coating the aid for eating or drinking or medical supply cured in step c. with an emulsion comprising carnauba wax, and/or canola wax, and/or kerosene, or mixtures thereof. Particularly preferred is an emulsion comprising carnauba wax which additionally comprises natural latex.

In a preferred embodiment, the emulsion of optional step d. of the manufacturing process consists of either 32.5% carnauba wax, 17.5% kerosene and 50% water, or 50% carnauba wax and 50% water as a wax-based coating, or 5% carnauba wax, 45% rubber and 50% water, or 5% carnauba wax and 95% natural latex as a natural latex-based coating. Optionally, excipients such as dyes or odor neutralizers and/or “anti-stick” additives, such as glycerin, may be added to the emulsion.

### Advantages of the Invention

Through the use of vegetable starch and vegetable thickening and gelling agents, the aids for eating or drinking and the medical supplies as disclosed herein are recyclable, fully biodegradable in addition to being compostable, unlike conventional disposable solutions such as plastic drinking straws, plastic cutlery or plastic ear swabs. An exemplary composting time of a drinking straw according to the invention, which was produced from a mixture comprising 50% starch, 45% guar gum 5% xanthan gum and, based on the net weight of these components, 2% carnauba wax emulsion, 40% water and 4% oil, was tested and found as food safe by a competent German authority (DEKRA). Furthermore, the drinking straw was evaluated and tested for compostability. The test resulted in a composting time of the drinking straw as less than 4 weeks in terms of intensive degradation, and a composting time of 27 days in dry fermentation. These values are far below those associated with alternative drinking straws. Any aid or supply of the present invention comprising these components is accordingly compostable within these time periods. An optional coating can extend the composting time by only a few days, so that even coated auxiliary or commodity products of the present invention are compostable within 4-8 weeks. Furthermore, the aids and supplies as disclosed herein are compostable according to International Standard (ISO) EN 13432, version 2000-12.

In some embodiments, the aids for eating or drinking and the medical supplies comprise cellulose, which increases the mechanical stability of the aids and the medical supplies, and also may prevent these products from buckling or tearing. The aids and medical supplies of the present invention comprise up to 15% cellulose. A higher cellulose content can render the aids or supplies as brittle. Particularly good mechanical properties are obtained at a cellulose content of 2-10%.

In some embodiments, the aids for eating or drinking and the medical supplies of the invention comprise wood pulp. Like cellulose, the wood pulp also increases mechanical stability. Compared to wood pulp, cellulose has the advantage that the aids or supplies have a higher transparency.

In some embodiments, the present aids for eating and drinking and medical supplies comprise wax, preferably carnauba wax or soy wax. Wax was shown to increase the resistance of the aids and the medical supplies to water and oils. The highly resistant carnauba wax can increase stability during use in a liquid by at least 10%, at least 20%, at least 30%, or even more than 30%. The use of soy wax also exhibits this stabilizing effect and is preferred.

In some embodiments, the aids for eating or drinking and the medical supplies of the present invention comprise oil, preferably nut oil. The oil improves the flow properties of the crude composition for the aids for eating or drinking and the medical supplies during the manufacturing process.

The proportion of vegetable thickening and gelling agents likewise influences the transparency of the material. The higher the proportion of vegetable thickening or gelling agent, the higher the material transparency.

In some embodiments, the aids for eating or drinking and the medical supplies of the invention comprise magnesium stearate and/or calcium stearate and/or zinc stearate. In this regard, magnesium stearate and calcium stearate are used as lubricants, non-stick agents, and gelling agents for improved flow properties of the raw material during manufacture. The addition of zinc stearate improves the absorption of the crude mass in devices used in the manufacturing process, e.g., the drawing-in of the crude mass by means of a plasticizing screw. In some embodiments, a portion of the added water is replaced with glycerin E422 during the manufacturing process to increase the mobility or flexing properties of the aids and medical supplies of the invention.

Drinking straws comprising an outer and inner coating of carnauba wax and rubber are stable for up to six days when immersed in water, while still being biodegradable and compostable: in garden compost, the drinking straws of the invention can biodegrade in less than 50 days, preferably less than 30 days. In industrial composting systems, the drinking straws were no longer detected in less than 14 days and were thus completely biodegradable. This biodegradable quality is also present in the other aids according to the invention having the same or similar composition.

The drinking straws according to the invention were further subjected to a food contact test conducted at an official testing center (DEKRA e.V.). These materials passed the food contact test in that neither heavy metals, nor pesticides, nor other harmful substances such as glyphosate were detected in the straws. These test results were also observed in other of the disclosed aids according to the invention having the same or similar composition.

Furthermore, the coating according to the invention imparts further advantageous properties to the drinking straw or the aids. The coating produces less friction on the user eating or consuming, for example, on the lips of the user; sticking of the drinking straw or the aids described herein can thus also be avoided.

The addition of odor neutralizers to the aids or medical supplies leads to odor neutralization thereby preventing any undesireable odors that may be present.

The addition of one or more “anti-stick” additives, also “anti-sticking agents” or “anti-tack” additives, or “anti-tack agents” to the coating emulsion prevents the aids or the medical supplies according to the invention from sticking to one another during storage. Furthermore, the addition of one or more anti-stick additives prevents the coating of an aid for eating or drinking from being rubbed off by friction, which can occur when plastic films are punctured.

### BRIEF DESCRIPTION OF THE DRAWINGS

FIG. 1 shows exemplary drinking straws according to the invention, wherein in some embodiments, the straws are colored with various auxiliary substances including dyes.

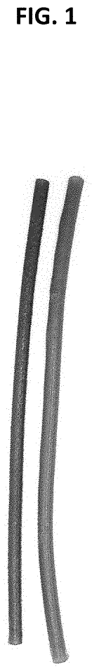

FIG. 2 provides an enlarged view of drinking straws according to the invention, which are not colored.

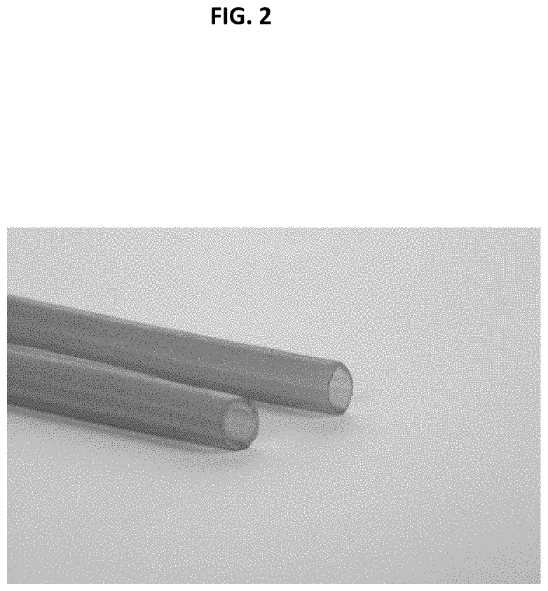

FIG. 3 illustrates lollipops with lollipop handles/lollipop sticks according to the invention. The lollipop sticks are provided in color.

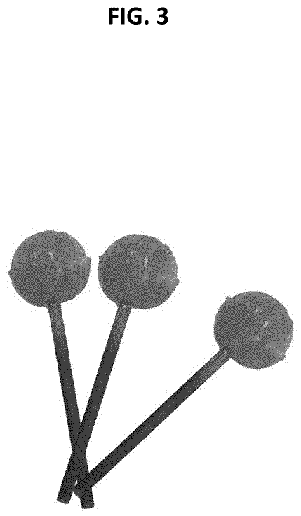

### DETAILED DESCRIPTION

### Definitions

“Aids for eating or drinking” as used herein refers to drinking straws, drinking cups, cutlery, lollipop handles/lollipop sticks, chopsticks, and popsicle sticks.

“Drinking straw” and “straw” are used interchangeably herein.

“Cutlery” is a collective term for various tools used to serve, cut and eat foodstuffs. Accordingly, cutlery comprises knives, forks, french fry forks, tablespoons, teaspoons, cake forks, ice cream spoons, and variations thereof.

“Medical supplies” refer to, for instance, cotton swabs, ear swabs, oral spatulas or tongue depressors, disposable toothbrushes, and disposable tongue cleaners.

“Vegetable starch” refers to any starch obtained from plant material. The starch may be obtained from, for example, roots, beets, tubers, rhizomes, shoot axes, leaves, fruits or seeds. Exemplary plant starches include wheat starch, potato starch, corn starch, or tapioca starch; starch from cassava (Manihot esculenta), tuber bean (Pachyrhizus tuberosus), batata (Ipomoea batatas), yam (Dioscorea spec.), tuberous pea (Lathyrus tuberosus), arakacha (Arracacia xanthorrhiza), tuberous wood sorrel (Oxalis tuberosa), tuberous nasturtium (Tropaeolum tuberosum), ulluco (Ullucus tuberosus), East Indian arrowroot (Tacca leontopetaloides), arrowroot (Maranta spec.), Achira (Canna indica), Taro (Colocasia esculenta), Tannia (Xanthosoma sagittifolium), White Water Lily (Nymphaea alba), Yellow Pond Lily (Nuphar lutea), or Chayote (Sechium edule).

“Fiber pulp” refers to fiber-derived material used in the manufacture of paper and paperboard. Fiber materials are composed mostly of cellulose. Fiber materials include cellulose and wood pulp.

“Cellulose” refers to the fibrous mass produced during the chemical pulping of plants, primarily wood. It consists to a large extent of cellulose. For the drinking straws of the present invention, cellulose having short fibers is preferably used. A particularly preferred fiber length is 0.8 mm to 1.1 mm. The cellulose of the present invention may be derived from many different plants, such as conifers, deciduous trees or bamboo. In particularly preferred embodiments, the cellulose is at least partially derived from baobab plant material. In another particularly preferred embodiment, the cellulose is at least partially derived from bamboo plant material.

“Wood pulp” refers to the fibrous mass produced during mechanical pulping of plants, especially wood. Wood pulp, unlike cellulose for higher quality papers, contains large amounts of lignin. For the aids for eating or drinking of the present invention, wood pulp with short fibers is preferably used. A particularly preferred fiber length is 0.8 mm to 1.1 mm. In some embodiments of the present invention, a combination of wood pulp and cellulose is used.

“Vegetable thickeners” and “vegetable gelling agents” are thickeners and gelling agents derived from vegetable or bacterial resources. Preferred are thickening and gelling agents from plant resources. They can cause the gelation of liquid. Exemplary vegetable thickening or gelling agents are agar-agar, pectin, carrageenan, alginates, locust bean gum, guar gum, sago, xanthan gum, gum arabic, rice flour, durum wheat flour or durum wheat semolina.

“Guar gum” (as food additive E412) is a vegetable thickener or gelling agent. It is obtained from ground seeds of the guar plant. It is particularly noteworthy that guar gum significantly enhances the effect of other vegetable thickening or gelling agents and is therefore popular for use with other vegetable thickening or gelling agents.

“Xanthan gum” (as food additive E415, also known as “xanthan”), is a bacterial thickening and gelling agent. It is produced by bacteria of the genus Xanthomonas from sugar-containing substrates. Preferably, the xanthan gum of the present invention is derived from bacteria of the species Xanthomonas campestris.

“Agar-agar” refers to a galactose polymer derived from algae. Agar-agar is obtained from the cell walls of algae, particularly red algae.

“Pectin” (as food additive E440a or E440b) refers to plant polysaccharides essentially comprising α-1-4-glycosidically linked galacturonic acids. They can be obtained, for example, from peels of apples, lemons and other fruits. Pectin is a vegetable thickening or gelling agent and to thereby ensure the gelling of a liquid.

“Carrageenan” (as food additive E407) is a vegetable thickener or gelling agent derived from various species of red algae.

“Alginate” is a vegetable thickening or gelling agent and consists of salts of alginic acid. It can be extracted from dried and ground brown algae. Depending on the salt, alginate is known by the E numbers E401, E402, E403, E404 and E405.

“Locust bean gum” (as food additive E410) is a vegetable thickener or gelling agent obtained from locust bean tree seeds by grinding. The flour obtained is white and tasteless.

“Sago” is a vegetable thickener or gelling agent. Sago is obtained from the starch-rich pulp of various plant species such as sago palm, cassava, or potatoes. It is often sold as granules in the form of small balls. Sago swells about threefold in hot liquid and has a strong binding effect when cooled. To prevent the liquid from becoming mushy, sago is only boiled or soaked until the globules are soft but still retain their shape.

“Gum arabic” (as food additive E414) is a vegetable thickener and gelling agent obtained from the resinous sap of acacia species native to Africa, such as Acacia senegal. Gum arabic can be in both powder and gum form.

“Rice flour” is a vegetable thickener and gelling agent produced by destemming rice grains and then finely grinding them. Depending on the use of polished or unpolished (brown) rice grains, white or brown rice flour is obtained.

“Durum wheat flour” is a vegetable thickener and gelling agent obtained from durum wheat (Triticum durum), also known as durum, durum wheat or glass wheat. The durum grains are hulled and then ground several times to produce durum wheat flour.

“Durum wheat semolina” is a vegetable thickener extracted from durum wheat (Triticum durum), also known as durum, durum wheat or glass wheat. As with durum wheat flour production, the durum grains are hulled and then milled. However, the durum grains are milled less intensively than in durum wheat flour production, thus durum semolina has a coarser particle structure.

“Wax” refers to any naturally occurring wax, such as carnauba wax, beeswax, or soy wax. Preferably, the wax in the present invention is carnauba wax. Preferably, biopolymers such as rubber have been added to the wax of the present invention. “Carnauba wax” is obtained from the leaf of the carnauba palm (Copernicia prunifera). The leaves are harvested and by drying and mechanical action the wax is separated. The wax is used in various industries, such as food, cosmetics and pharmaceuticals. Untreated carnauba wax has a light yellowish, greenish to dark gray color, it is interspersed with air bubbles, hard, brittle and insoluble in water. Carnauba wax is the hardest natural wax with the highest melting point of over 80° C. It is also edible; thus it is naturally food-grade, and has a mild taste. This is advantageous for drinking straws and cutlery that includes carnauba wax, as the drinking straws and cutlery thus have no inherent taste and are naturally a food-grade product. Moreover, the addition of carnauba wax to the other materials such as vegetable starch, agar-agar and cellulose improves the manufacturing process of the materials, while maintaining the strength, hardness and resistance of the drinking straws and cutlery to be obtained. In the present invention, carnauba wax is preferably used in powder form. In particular embodiments, biopolymers such as rubber are added to the above wax.

“Soy wax” is obtained from ripe soybeans (Glycine max). The soybeans are first harvested, and soybean oil is obtained. The oil is hydrogenated, under pressure of about 200 bar and temperatures of about 140° C.-225° C. in the presence of a metallic catalyst, where soy wax is obtained. The wax is used in various industries, such as cosmetics and candles. Untreated soy wax has a very light, creamy white, sometimes yellowish color, is hard, brittle and insoluble in water. Soy wax has a melting point of about 50° C. It is also edible and has a mild taste. This is advantageous for drinking straws and cutlery, as the straws and cutlery thus have no inherent taste and represent a food-grade product without any further processing step. In addition, the addition of soy wax to the other materials such as vegetable starch, agar-agar and cellulose makes the processing of the materials smoother, while maintaining the strength, hardness and resistance of the drinking straws and cutlery to be obtained. In the present invention, soy wax is preferably used in powder form. In particular embodiments, biopolymers such as rubber are added to the above wax. “Canola wax” is obtained from the seeds of canola (Brassica napus) or also from the close relative oilseed rape (Brassica rapa subsp. oleifera). The seeds are harvested, and a first canola oil is extracted. By hardening canola oil, canola wax is obtained. The melting point of canola wax is approximately between 57° C. and 61° C. Canola wax is also edible. “Carnauba wax emulsion” describes an emulsion based on carnauba wax comprising 30-60% (carnauba wax) solids content and 70-40% water and optionally a small amount of ammonia (0.03%), preferably the carnauba wax emulsion contains 50% carnauba wax and 50% water, optionally 0.03% ammonia. Optionally included ammonia reduces settling of the solid portions of the emulsion.

“Biopolymer” refers to a polymer based on renewable raw materials that is (biologically) degradable. An exemplary biopolymer is rubber, also called gum eleasticum or resina elastica. Rubber offers a higher resistance to water than comparable resins. Therefore, rubber itself or blends thereof are particularly advantageous. Rubber is used in the form of natural latex, alternatively in the form of an emulsion based on rubber and water with a solids content of 40-60%, preferably 50-65%, most preferably 60% rubber. Certain petroleum-based polymers are also biodegradable and therefore biopolymers. Petroleum-based polymers of the invention include polyvinyl alcohol (PVA), polybutylene adipate terephthalate (PBAT), polybutylene succinate (PBS), polycaprolactone (PCL), and polyglycolide (PGA). The biopolymers of the present invention can also be modified and are thus “bio-based polymers”. These include polylactide (PLA), polyhydroxyalkanoates (PHA), polyhydroxybutyrate (PHB), lignin-based materials such as thermoplastics.

“Emulsion” means any distributed mixture of several components, such as one or more solids and a solvent such as water. An emulsion may be a wax emulsion, a carnauba wax emulsion, or rubber emulsion. For example, an emulsion according to the invention comprises carnauba wax, kerosene, and water. Another emulsion according to the invention comprises carnauba wax, rubber and water. Natural latex is also an emulsion of rubber and water.

“Oil” means any vegetable, mineral or animal oil. Preferably, the oil in the present invention is a vegetable oil. Nut oil and sunflower oil are particularly preferred.

“Compostable” refers to the property of a material to be 90% degraded after 6 months under defined aerobic conditions. The compostability of a material can be determined according to German testing standard procedures DIN EN 13432, version 2000-12. Compostability herein refers to both industrial composting and non-industrial composting by purely biological decomposition or rotting (home composting). Thus, the aids and medical supplies according to the invention are compostable both by means of inventive rotting and by means of dry fermentation within 4-8 weeks, preferably within 4 weeks, such that the aids and supplies have completely disintegrated within this time.

“Biodegradable” refers to the characteristic of a process to completely degrade an organic substance biologically, that is, by living organisms or their enzymes. In this process, the degradation of the substance takes place under aerobic conditions in 10 years or less, preferably in 5 years, even more preferably in one year.

“Recyclable” means that after the material has been used (for example, as a drinking straw), the material can be used through a reprocessing (recycling) process as a material to produce a new product that is not intended for incineration. This is in contrast to materials that, after use, must either be recovered through incineration or permanently landfilled. Recyclable materials include cellulose, paper, paperboard and cellulose hydrate.

“Magnesium stearate” Magnesium stearate is the magnesium salt of stearic acid and is a member of the lime soap classification. It is obtained from fats and oils by splitting their glycerides using magnesium, soaps and glycerin. Magnesium stearate is used, for example, in the pharmaceutical industry as an auxiliary ingredient in tablet or granule production. Magnesium stearate is also used to produce candies/sweets. Magnesium stearate can be produced from fats of both animal and vegetable origin. Soybean, canola or corn germ oil is commonly used. This substance is also useful because it has lubricating properties that can prevent the ingredients from sticking to manufacturing equipment during compression of chemical powders into solid tablets. Magnesium stearate is the most commonly used lubricant for tablets. In the manufacture of pressed candies, magnesium stearate acts as a release agent and is used to bind sugars in hard candies such as mints. Magnesium stearate is commonly used as a lubricant at concentrations ranging from 0.25% to 5.0% in the manufacture of tablets, capsules and other oral dosage forms. Due to its long-standing use as an excipient in the pharmaceutical, food and cosmetic industries, the safety of magnesium stearate is well documented.

“Calcium stearate” Calcium stearate is used for the production of so-called non-toxic stabilizers of plastics, preferably in combination with zinc stearate, but also barium stearate or magnesium stearate. It is also used as a lubricant in pharmaceutical products and as a lubricant (buffer grease) in the paper and metal processing industries, as a water repellent for building materials and in sand processing. Technical calcium stearate is obtained by reacting calcium chloride with the sodium salt of stearic acid (usually contaminated with the sodium salt of palmitic acid) and then washing out sodium chloride. It is also an impregnating agent for textiles. In plastics, it can act as an acid scavenger or neutralizer in concentrations up to 1000 ppm, as a lubricant, and as a release agent. It can be used in plastic colorant concentrates to improve pigment wetting. In rigid PVC, it can accelerate fusion, improve flow and reduce stamp swelling. Applications in the personal care and pharmaceutical industries include tablet mold release agents, non-stick agents and gelling agents. Calcium stearate is a component of some types of defoamers. In addition, calcium stearate is an anti-caking agent that allows the fine mobility of solids, preventing powdered components from caking together. In papermaking, calcium stearate is used as a lubricant to achieve good gloss, and to prevent dusting and wrinkle cracking in paper and board production. An addition of about 0.1 to 10% is feasible.

“Zinc stearate” is a white powder with a melting point of 130° C., a flash point of 277° C. and a self-ignition temperature of 420° C. The molecular weight is 632.3 g/mol. Zinc stearate is not soluble in water. Zinc stearate is used as a stabilizer for emulsions. During card tricks, zinc stearate is commonly used as a card powder, which improves the gliding properties of playing cards. Zinc stearate is also used as a lubricant in plastics processing. It prevents polyamide parts from sticking to each other and is an aid in plasticizing problems. If the plasticizing screw herein does not draw in the material properly, this can be improved by adding about 0.2% zinc stearate, especially for polyamide 6.0. If the plasticizing screw does not draw in the material properly, this can be improved by adding about 0.2% zinc stearate.

“Glycerin” Glycerin is the trivial name and common designation of propane-1,2,3-triol, a fatty substance used in the present invention as a lubricant and humectant. Preferred is E 422. E 422 is obtained predominantly from vegetable fats and oils. Production from synthetic or animal substances is also possible. Preferred is “vegetarian glycerin” or “vegan glycerin”, which is produced by the transesterification of vegetable oils. As a food additive, E 422 is generally approved for all foods and there is also no maximum amount restriction for the use of glycerin. In the present invention, the addition of glycerin increases the mobility of the material, which may be particularly useful in the drinking straws of the invention.

“Excipients” are substances that impart further advantageous properties to the present invention, for example in terms of shape, manufacturability, stability. Excipients include, for example, antioxidants, binders, emulsifiers, stabilizers, colorants, fragrances or odor neutralizers and/or brighteners. Preferably, these excipients are biodegradable. Odor neutralizers include substances from vanilla, lemon lavender or fir. Fragrances or odor neutralizers are preferably comprised in the coating of the aids and supplies according to the invention and are preferably added to a coating emulsion in a ratio of 1 L coating emulsion:1 teaspoon odor neutralizer (powder or liquid).

“Rubber” refers to natural rubber present as an emulsion with water, as well as natural latex or natural latex milk, such as natural latex Laguna from the company Colok GmbH. The latex or rubber emulsion may additionally comprise a release agent such as Struktol® or ammonia. The preferred solids content of a rubber emulsion is 60% rubber. Natural latex and rubber emulsion refer to the same emulsion and are to be understood interchangeably.

“Anti-stick additives”, also referred to as “anti-sticking agents” or “anti-tack” additives, or “anti-tack agents” prevent products according to the invention from sticking to each other when several of such articles according to the invention are in direct contact with each other during storage, such as in storage containers. The terms are known to those skilled in the art. The exact compositions of such anti-stick or anti-tack additives are generally manufacturer-specific. Anti-tack additives are preferably used with rubber emulsion or latex. Anti-stick additives include oils, glycerin, lecithins, vegetable fats and vegetable stearates.

Any additives and components of the present invention are approved for use in food and are purely vegetable based.

### Embodiments of the Invention

The aids for eating or drinking, as well as the medical supplies of the present invention may have different compositions. Invariably, they comprise vegetable starch and a vegetable thickening or gelling agent. Most preferred is guar gum as a thickening or gelling agent, followed by xanthan gum, agar-agar, pectin, carrageenan, alginate, locust bean gum, sago, gum arabic, rice flour, durum wheat flour and durum wheat semolina. Further, any combination of the listed thickening or gelling agents may also be listed. For example, multiple vegetable thickening or gelling agents may be used in identical amounts (in a 1:1 ratio). The amounts of the multiple thickening and gelling agents may also be different. For example, in some embodiments, the ratio of two gelling agents may be between 10:1 and 1:10, preferably between 10:1 and 1:5, more preferably between 5:1 and 1:2. Exemplary ratios include 5:1, 4:1, 3:1, 2:1, and 1:2.

In some embodiments, the aids for eating or drinking and the medical supplies also comprise cellulose and/or wood pulp, which provides the advantages described above. Further, the aids for eating or drinking and medical supplies of the present invention may optionally comprise wax, preferably carnauba wax or soy wax, which also provides advantages as described above. The aids for eating or drinking and the medical supplies of the present invention may optionally comprise oil, preferably nut oil.

Embodiments Comprising Vegetable Starch and Agar-Agar

The following embodiments relate to aids for eating or drinking preferably drinking straws and cutlery comprising vegetable starch and agar-agar.

The vegetable starch content is 30-50%, preferably 35-50%, more preferably 35-45%. An exemplary starch content is 43%.

The vegetable starch is wheat starch, potato starch, corn starch, tapioca starch, or starch from cassava, tuberous bean, batata, yam, tuberous pea, arakacha, tuberous wood sorrel, tuberous nasturtium, ulluco, East Indian arrowroot, arrowroot, achira, taro, tannia, white water lily, yellow pond lily, or chayote, or a mixture thereof.

Preferably, the vegetable starch is wheat starch, potato starch, corn starch, tapioca starch, or a mixture thereof.

The agar-agar content is 30-60%, preferably 30-50%, more preferably 40-50%. An exemplary agar-agar content is 50%.

The cellulose content is 0-10%, preferably 1-10%, more preferably 2-10%. An exemplary cellulose content is 4%.

The wood pulp content is 0-10%, preferably 1-10%, more preferably 2-10%. An exemplary wood pulp content is 4%.

Cellulose and wood pulp may be used in combination. In this case, the total cellulose and wood pulp content is 0-10%, preferably 1-10%, more preferably 2-10%. An exemplary total content is 4%. The ratio of cellulose to wood pulp is between 10:1 and 1:10, preferably between 10:1 and 1:5, more preferably between 5:1 and 1:2. Exemplary ratios are 5:1, 4:1, 3:1, 2:1, 1:1 and 1:2. The cellulose and wood pulp preferably have fibers with a length of 0.8-1.1 mm.

The wax content, preferably carnauba wax, is 0-10%, preferably 1-10%, more preferably 1.5-7%. An exemplary wax content is 3%.

The content of oil, preferably nut oil, is 0-1.5%, preferably 0.1-1%, more preferably 0.2-0.7%. An exemplary oil content is 0.5%.

In preferred embodiments, the aids for eating or drinking of the present invention comprise 30-50% vegetable starch, 30-50% agar-agar 1-10% cellulose and/or 1-10% carnauba wax. In particularly preferred embodiments, the aids for eating or drinking of the present invention comprise 30-50% vegetable starch, 30-50% agar-agar, 1-10% cellulose, and 1-10% carnauba wax.

In an even more preferred embodiment, the aids for eating or drinking of the present invention comprise 43% vegetable starch, 50% agar-agar, 4% cellulose and 3% carnauba wax.

In an equally preferred embodiment, the medical supplies of the present invention comprise 30-50% vegetable starch, 30-50% agar-agar 1-10% cellulose and/or 1-10% carnauba wax. In particularly preferred embodiments, the medical supplies of the present invention comprise 30-50% vegetable starch, 30-50% agar-agar, 1-10% cellulose, and 1-10% carnauba wax.

In an even more preferred embodiment, the medical supplies of the present invention comprise 43% vegetable starch, 50% agar-agar, 4% cellulose and 3% carnauba wax.

In preferred embodiments, the aids for eating and for drinking include a drinking straw and/or cutlery. Therefore, the present invention also includes drinking straws and cutlery comprising 30-50% vegetable starch, 30-50% agar-agar 1-10% cellulose and/or 1-10% carnauba wax. Particularly preferred are drinking straws and/or cutlery comprising 43% vegetable starch, 50% agar-agar, 4% cellulose and 3% carnauba wax.

Embodiments Comprising Vegetable Starch and Vegetable Thickener or Gelling Agent, Preferably Guar Gum and Xanthan Gum

The following embodiments relate to aids for eating or drinking and medical supplies comprising vegetable starch and vegetable thickening or gelling agent, preferably guar gum and xanthan gum.

The vegetable starch content is 30-70%, preferably 50-65%, more preferably 50-60%. An exemplary starch content is 50%.

The vegetable starch can be wheat starch, potato starch, corn starch, tapioca starch, or starch from cassava, tuberous bean, batata, yam, tuberous pea, arakacha, tuberous wood sorrel, tuberous nasturtium, ulluco, East Indian arrowroot, arrowroot, achira, taro, tannia, white water lily, yellow pond lily, or chayote, or a mixture thereof.

Preferably, the vegetable starch is wheat starch, potato starch, corn starch, tapioca starch, or a mixture thereof.

The vegetable thickener and gelling agent content can be 30-70%, preferably 40-60%, more preferably 40-50%. An exemplary thickening and gelling agent content is 50%.

The vegetable thickening and gelling agent is guar gum, xanthan gum, agar agar, pectin, carrageenan, alginate, locust bean gum, sago, gum arabic, rice flour, durum wheat flour or durum wheat semolina, or a mixture thereof.

Preferably, the vegetable thickener and gelling agent is a mixture of guar gum and xanthan gum.

The aids for eating or drinking and the medical supplies may optionally comprise cellulose. The cellulose content is 0-10%, preferably 1-10%, more preferably 2-10%. An exemplary cellulose content is 4%.

The aids for eating or drinking and the medical supplies may optionally comprise wood pulp. The wood pulp content is 0-10%, preferably 1-10%, more preferably 2-10%. An exemplary wood pulp content is 4%.

Cellulose and wood pulp may be used in combination. In this case, the total cellulose and wood pulp content is 0-10%, preferably 1-10%, more preferably 2-10%. An exemplary total content is 4%. The ratio of cellulose to wood pulp is between 10:1 and 1:10, preferably between 10:1 and 1:5, more preferably between 5:1 and 1:2. Exemplary ratios are 5:1, 4:1, 3:1, 2:1, 1:1 and 1:2. The cellulose and wood pulp preferably have fibers with a length of 0.8-1.1 mm.

The aids for eating or drinking and the medical supplies may optionally comprise wax. The wax content, preferably carnauba wax or soy wax, is 0-10%, preferably 1-10%, more preferably 1.5-7%. An exemplary wax content is 3%.

The aids for eating or drinking and the medical supplies may optionally comprise oil. The oil content, preferably nut oil, at 0-4%, more preferably 0.1-3%. An exemplary oil content is 2%.

The aids for eating or drinking and the medical supplies may optionally comprise glycerin. The glycerin content can be 0-10%, preferably 0-3%. An exemplary glycerin content is 2%.

In another embodiment, the aids for eating or drinking of the present invention comprise 40-50% vegetable starch, 35-45% guar gum, and 1-5% xanthan gum.

Outer Coating

All of the aforementioned embodiments of the aids for eating or drinking and medical supplies according to the present invention comprising vegetable starch and vegetable thickener or gelling agent, preferably guar gum and xanthan gum, may further comprise an outer coating. The coating can comprise carnauba wax, and/or canola wax, and/or kerosene, or mixtures thereof. Preferably, biopolymers that are based on renewable raw materials and are (biologically) degradable, such as rubber or natural latex, or certain petroleum-based polymers that are biodegradable, such as polyvinyl alcohol (PVA), polybutylene adipate terephthalate (PBAT), polybutylene succinate (PBS), polycaprolactone (PCL), and polyglycolide (PGA), may be added to the wax. Alternatively, bio-based polymers such as polylactide (PLA), polyhydroxyalkanoates (PHA), polyhydroxybutyrate (PHB), or lignin-based materials such as thermoplastics may be included by the wax coating. Depending on the desired thickness, the coating may comprise 0-10%, preferably 0.5-2%, of the composition of the aid or medical supply.

In a preferred embodiment, the coating is a wax-based coating comprising wax as the main ingredient (wax layer). A preferred wax emulsion comprises 30-50% wax, preferably carnauba wax or canola wax, 50-70% water, and optionally excipients and/or one or more anti-stick additives. Preferably, the wax-based emulsion contains 32.5% carnauba wax, 17.5% kerosene and 50% water or consists of 32.5% carnauba wax, 17.5% natural latex and 50% water. Another preferred wax emulsion consists of 50% carnauba wax and 50% water. A natural latex-based coating is particularly preferred, which contains natural latex as the main ingredient. A natural latex-based emulsion consists of 50-95% natural latex 5-10% wax, preferably carnauba wax or canola wax, and optionally excipients and/or one or more anti-stick additives. Preferably, the natural latex-based emulsion contains 10% carnauba wax and 90% rubber emulsion consisting of 60% solid rubber and 40% water, or natural latex milk, or 5% carnauba wax, 45% rubber and 50% water, or of 5% canola wax, 45% rubber and 50% water, or of 5% carnauba wax and 95% natural latex, or of 5% carnauba wax, 2% glycerin, 93% natural latex.

In addition, the outer coating may include one or more anti-stick additives. The presence of such additives prevents subsequent sticking of the individual aids and supplies to each other when stacked or lined up or stored side by side. Anti-stick additives of the present invention are oils comprising canola oil, coconut oil and sunflower oil, preferably canola oils. The oil is added at 0-5%, preferably 0.5-1%, most preferably 0.5% of the amount of coating emulsion. Another anti-stick additive is sunflower lecithin or soy lecithin. This can be added in addition to an oil to help the oil mix with the wax emulsion. An additional anti-stick additive is a stearate, such as magnesium stearate of vegetable origin. This can be added to the wax emulsion in an amount of 0.25%-0.75%, preferably 0.3%. The optional anti-stick additive glycerin can either be added to the coating emulsion or sprayed as a kind of second coating onto the cured first coating by means of nozzles.

In one embodiment, the aids for eating or drinking of the present invention comprise:
40-50% vegetable starch,
35-50% vegetable thickener or gelling agent,
0-10% cellulose and/or wood pulp,
0-10% wax, preferably carnauba wax or soy wax,
0-3% nut oil or sunflower oil,
0-2% glycerin, and
optionally an outer coating.

In yet another embodiment, the aids for eating or drinking of the present invention comprise 65% vegetable starch, 30% guar gum and 5% xanthan gum and optionally an outer coating.

In another embodiment, the aids for eating or drinking of the present invention comprise 50% vegetable starch, 45% guar gum and 5% xanthan gum and optionally an outer coating.

In another preferred embodiment, the aids for eating or drinking of the present invention comprise:
40-50% vegetable starch,
35-45% guar gum,
1-5% xanthan gum,
0-10% cellulose and/or wood pulp,
0-10% wax, preferably carnauba wax or soy wax,
0-3% nut oil or sunflower oil,
0-2% glycerin,
optionally an outer coating.

In another more preferred embodiment, the aids for eating or drinking of the present invention comprise:
40-50% vegetable starch,
35-45% guar gum,
1-5% xanthan gum,
0-10% cellulose and/or wood pulp,
0.5-10% wax, preferably carnauba wax and/or canola wax,
0.1-3% nut oil or sunflower oil,
0.1-2% glycerin,
optionally, an outer coating.

In another embodiment, the aids for eating or drinking of the present invention comprise:
42% vegetable starch comprising wheat starch, corn starch and/or tapioca starch,
42% thickening or gelling agents comprising guar gum and xanthan gum,
2% wax, preferably carnauba wax and/or canola wax,
3% nut oil or sunflower oil,
2% glycerin, and
9% wax layer comprising carnauba wax, and/or canola wax, and/or kerosene. Particularly preferred is a wax layer comprising carnauba wax, which additionally contains rubber.

In another embodiment, the aids for eating or drinking of the present invention, preferably drinking straws, comprise:
50% starch,
5% xanthan gum,
45% guar gum, and
colorant
and a coating,
wherein the coating emulsion consists of 5% carnauba wax, 2% glycerin, 43% natural latex and 50% water.

This embodiment may include residues of glycerin and oil added as liquid components during the manufacturing process.

The embodiments herein relate to any aid for eating or drinking. Accordingly, the embodiments relate to drinking straws, drinking cups, cutlery, lollipop sticks, sushi sticks, popsicle sticks, etc.

Further, the embodiments relate to medical supplies such as ear swabs, cotton swabs, disposable toothbrushes, and tongue depressors.

In another preferred embodiment, the medical supplies of the present invention comprise:
40-50% vegetable starch,
35-45% guar gum,
1-5% xanthan gum,
0-10% cellulose and/or wood pulp,
0-10% wax, preferably carnauba wax and/or soy wax,
0-3% nut oil or sunflower oil,
0-2% glycerin,
and optionally an outer coating.

In another more preferred embodiment, the medical supplies of the present invention comprise:
40-50% vegetable starch,
35-45% guar gum,
1-5% xanthan gum,
0-10% cellulose and/or wood pulp,
0.5-10% wax, preferably carnauba wax and/or canola wax,
0.1-3% nut oil or sunflower oil,
0.1-2% glycerin,
and optionally an outer coating.

In another embodiment, the medical supplies of the present invention comprise:
42% vegetable starch comprising wheat starch, corn starch and/or tapioca starch,
42% thickening or gelling agents comprising guar gum and xanthan gum,
2% wax, preferably carnauba wax and/or canola wax,
3% nut oil or sunflower oil,
2% glycerin, and
9% wax layer comprising carnauba wax, and/or canola wax, and/or kerosene. Particularly preferred is a wax layer comprising carnauba wax, which additionally contains rubber.

Properties of the Aids and Medical Supplies

The aids for eating or drinking and the medical supplies of the present invention are biodegradable and/or even compostable. In some embodiments, an aid/medical supply is opaque. In other embodiments, an aid/medical supply is semi-transparent. The color of the aids for eating or for drinking/medical supplies can be modified using conventional dyes, preferably food coloring.

Aids for drinking—drinking straw: The wall thickness of the drinking straw is variable. The wall of the drinking straw can have a thickness between 0.1 mm and 2 mm, preferably between 0.3 mm and 1.5 mm or between 0.5 mm and 1.3 mm. Exemplary wall thicknesses here are 1 mm and 1.2 mm. The diameter of the drinking straw is variable. The diameter of the drinking straw can be between 1 mm and 3 cm, preferably between 3 mm and 1.5 cm or between 5 mm and 8 mm. Exemplary diameters here are 6 mm and 7 mm. The length of the drinking straw is variable. The length of the drinking straw can be between 5 cm and 50 cm, preferably between 10 cm and 35 cm or between 15 cm and 30 cm. Exemplary lengths as disclosed herein are 20 cm, 21 cm and 25 cm. In one embodiment, the drinking straw further includes preservatives to prevent spoilage of the biodegradable material. The preservative content is 0-2%, preferably 0.2-1%, more preferably 0.5%.

In one embodiment, the drinking straw additionally includes glycerin E 422 (vegan/vegetarian glycerin) to increase the mobility or flexing property of the drinking straws of the invention.

The drinking straws of the present invention are suitable for a variety of uses. They are suitable for a variety of beverages, such as hot beverages, tea, coffee, cold beverages, such as juice, juice spritzers, sodas, water, etc., or alcoholic beverages, such as beer, wine, cocktails, etc.

Aids for eating—cutlery: The thickness of the cutlery disclosed herein is variable. The thickness of the cutlery can be between 1 mm and 20 mm, preferably between 3 mm and 17 mm or between 5 mm and 15 mm. An exemplary thickness is 10 mm.

In one embodiment, the cutlery further includes preservatives to prevent spoilage of the biodegradable material. The preservative content is 0-2%, preferably 0.2-1%, more preferably 0.5%.

The cutlery of the present invention is suitable for a variety of uses. It is suitable for a variety of foods, such as hot dishes, soups, stews, casseroles, meat and fish dishes, side dishes such as potatoes, as well as cold dishes such as salads, ice cream and yogurt.

Aids for eating—lollipop handle/lollipop stick: The dimensions of a lollipop handle/lollipop stick of the present invention are variable. The diameter can range from 1 mm to 4 mm. The wall thickness can range from 0.5 to 1.5 mm. In some embodiments, the stick is not hollow. The length may be between 10 and 15 cm. In a preferred embodiment, a lollipop handle/lollipop stick has a diameter of 4 mm, a wall thickness of 0.5 mm, and a length of 12 cm. In some embodiments, a hole is pierced in the wall of the end portion of the stick that carries the sugar mass. This serves to hold the sugar mass in place.

Medical supplies—ear swabs: The dimensions of an ear swab (or cotton swab) of the present invention are variable. The diameter may range from 1 mm to 4 mm. The wall thickness may range from 0.5 to 1.5 mm. In some embodiments, the stick is not hollow. The length may be between 10 and 15 cm. In a preferred embodiment, an ear swab has a diameter of 4 mm, a wall thickness of 0.5 mm, and a length of 10 cm.

Manufacturing Methods

The present invention also provides methods for the production of aids for eating or drinking in accordance with the present invention. In this regard, the method comprises the following steps:
a. Preparing a mixture comprising vegetable starch and vegetable thickening or gelling agent, comprising the steps of
   a.1 combining solid components of the mixture comprising vegetable starch and vegetable thickening or gelling agent and mixing same,
   a.2 adding liquid components comprising water to the mixture of step a.1 in an amount of 30-60% of the net weight of the mixture of a.1 and mixing same,
b. forming the mixture from step a. into aids for eating or drinking,
c. curing the aids for eating or drinking formed in step b.,
d. optionally, coating the aids for eating or drinking cured in step c. with an emulsion comprising carnauba wax, and/or canola wax, and/or kerosene, or mixtures thereof. Particularly preferred is a coating comprising carnauba wax which additionally comprises rubber.

The present invention also provides methods for the production of medical supplies in accordance with the present invention. In this regard, the method comprises the following steps:
a. Preparing a mixture comprising vegetable starch and vegetable thickener or gelling agent, comprising the steps of
   a.1 combining solid components of the mixture comprising vegetable starch and vegetable thickening or gelling agent and mixing same,
   a.2 adding liquid components comprising water to the mixture of step a.1 in an amount of 30-60% of the net weight of the mixture of a.1 and mixing same,
b. forming the mixture from step a. into medical supplies,
c. curing the medical supplies formed in step b.,
d. optionally, coating the medical supplies cured in step d. with an emulsion comprising carnauba wax, and/or canola wax, and/or kerosene, or mixtures thereof.

Step a:

In step a., vegetable starch and thickening or gelling agents are mixed with water. This can be done with a blender that mixes the mixture into a homogeneous mass. The mixture may optionally also include cellulose, wood pulp, wax (preferably carnauba wax or soy wax), and oil (preferably nut oil), glycerin, and/or a wax emulsion. The procedure can be as follows:

In step a.1, the solid components comprising vegetable starch and thickening and gelling agents are first mixed well together in a dry, powder state. This is important to counteract lump formation when mixing with water.

The vegetable starch in the mixture in step a.1 can be wheat starch, potato starch, corn starch, tapioca starch, or starch from cassava, tuber bean, batata, yam, tuber vetch, arakacha, tuberous wood sorrel, tuberous nasturtium, ulluco, East Indian arrowroot, arrowroot, achira, taro, tannia, white water lily, yellow pond lily, or chayote, or a mixture thereof.

Preferably, the vegetable starch in the mixture in step a.1 is wheat starch, potato starch, corn starch, tapioca starch, or a mixture thereof.

Preferably, the vegetable thickening and gelling agent in the mixture in step a.1 is guar gum, xanthan gum, agar agar, pectin, carrageenan, alginate, locust bean gum, sago, gum arabic, rice flour, durum wheat flour or durum wheat semolina, or a mixture thereof.

Preferably, the vegetable thickener and gelling agent is a mixture of guar gum and xanthan gum.

In some embodiments, the solid components in step a1. further comprise cellulose and/or wood pulp. The cellulose may be at least partially made from baobab plant material or bamboo plant material.

The proportions of the components in the total amount of the mixture of step a.1 are described below:

The amount of plant starch is 30-70%, preferably 50-65%. An exemplary amount of starch is 50%.

The amount of vegetable thickener and gelling agent is 30-70%, preferably 35-50%. An exemplary vegetable thickener and gelling agent amount is 50%.

The amount of cellulose is 0-10%, preferably 1-10%, more preferably 2-10%. An exemplary cellulose amount is 2%.

The amount of wood pulp is 0-10%, preferably 1-10%, more preferably 2-10%. An exemplary wood pulp amount is 2%.

Cellulose and wood pulp may be used in a combination. In this case, the total amount of cellulose and wood pulp is 0-10%, preferably 1-10%, more preferably 2-10%. An exemplary total amount is 2%. The ratio of cellulose to wood pulp is between 10:1 and 1:10, preferably between 10:1 and 1:5, more preferably between 5:1 and 1:2. Exemplary ratios are 5:1, 4:1, 3:1, 2:1, 1:1 and 1:2. The cellulose and wood pulp preferably have fibers with a length of 0.8-1.1 mm.

In a preferred embodiment, the mixture in step a.1 of the present invention comprises:
50-70% vegetable starch, and
30-50% vegetable thickener or gelling agent.

In an even more preferred embodiment, the mixture in step a.1 of the present invention comprises:
65% vegetable starch,
30% guar gum, and
5% xanthan gum.

In another preferred embodiment, the mixture in step a.1 of the present invention comprises:
50% vegetable starch,
45% guar gum, and
5% xanthan gum.

The amount of vegetable starch, thickener or gelling agent and cellulose/wood pulp thus sums up to a total amount of the mixture of step a.1 of 100%. The other components of the mixture from step a (total mixture), such as additional solid components including wax powder or the liquid components from step a.2, are calculated on the basis of net weight of the mixture of solid components from step a.1 and are therefore indicated as the % of the net weight of the mixture from step a.1.

Additional solid components may be included in step a.1. In some embodiments, the solid components in step al further comprise wax powder, preferably carnauba wax powder, and/or canola wax powder, and/or soy wax powder.

The amount of wax powder, preferably carnauba wax or soy wax, is 0-10% of the net weight of the solid components from the mixture in step a.1, preferably 1-10%, more preferably 1.5-4% of the net weight of the solid components from the mixture in step a.1. An exemplary amount of wax is 3% of the net weight of the solid components from the mixture in step a.1. The following example will explain the calculation for 3% wax powder: Given a net weight of the mixture of vegetable starch, vegetable thickener or gelling agent and optionally cellulose/wood pulp of 1000 g, 30 g of wax powder is added to this mixture. The components are then mixed well.

In another embodiment, the mixture in step a.1 of the present invention comprises:
55% vegetable starch,
45% guar gum,
5% xanthan gum, and
based on the net weight of this mixture
1% carnauba wax powder.

In step a.2, the liquid components are added to the mixture from step a.1 in an amount based on the net weight of the mixture from a.1 in mL and mixed well. The liquid components include water and optionally other liquid components such as a wax emulsion, glycerin, and/or oil.

A preferred wax emulsion consists of 30-50% solids and 50-70% water, preferably 40% solids and 60% water. This emulsion is heated with continuous stirring until the solid liquefies and then immediately added to the mixture from a.1. The following example illustrates this calculation: For an addition of wax emulsion of 2% of the net weight of the mixture from a.1, the volume of the wax emulsion is 20 mL for a net weight of the mixture from a.1 of 1000 g. The wax includes any food grade wax such as carnauba wax, soy wax, beeswax, canola wax, food grade kerosenes, or a mixture thereof. Preferred is an emulsion of carnauba wax, and/or soy wax, and/or canola wax.

Optionally, oil, preferably nut oil or sunflower oil is added to the mixture of a.1. The amount of oil added is 1-5% of the net weight of the mixture of a.1 in mL, preferably 2-4% of the net weight of the mixture of a.1 in mL, more preferably 4% of the net weight of the mixture of a.1 in mL. The following example will illustrate the calculation: For a net weight of the mixture of a.1 of 1000 g, 20-40 mL of oil is added to this mixture. Then the components are again mixed thoroughly.

Optionally, food coloring, preferably in liquid form, is added to the mixture from a.1. The amount of food coloring can replace 1-50% of the added water.

The temperature of the mass during the mixing process is between 30 and 40° C., preferably 35° C.

Step a.2 comprises by way of example:
a.2 addition of liquid components of the mixture to the mixture of step a.1 comprising, based on the net weight of the mixture of step a.1:
   2% carnauba wax emulsion,
   4% sunflower oil,
   5% glycerin, and
   40% water,
   and mixing of same

Table 1 shows preferred mixtures according to step a, for instance, according to steps a.1 and a.2.

| | Mixture of step a comprising: | | | | | | | |
|---|---|---|---|---|---|---|---|---|
| | Solid components (mixture of step a.1) | | | | | | | |
| | | Vegetable | | Solid components | | | | |
| | | thickening- | | (as a % of the | Liquid components (as a % of the | | | |
| | Vegetable | and gelling | | net weight of | net weight of the mixture of | | | |
| | starch (e.g., | agent (e.g., | | the mixture of | solid components of a.1, in mL) | | | |
| | from wheat, | guar gum, | | solid components | Oil (e.g., | | | |
| Mixture | corn, potato, | xanthan gum, | Cellulose/ | of a.1, in grams) | sunflower oi, | | Wax | |
| of step a | tapioca) | agar agar) | Wood pulp | Wax (powder) | nut oil) | Glycerin | emulsions | Water |
| 1 | 50% | 50% | | | 4% | 5% | | 40% |
| | corn- | agar agar | | | | | | |
| | starch | | | | | | | |
| 2 | 70% | 30% | | | 4% | 10% | | 40% |
| 3 | 60% | 40% | | | 4% | 8% | | 40% |
| 4 | 55% | 45% | | | 4% | 6% | | 40% |
| 5 | 50% | 49% | 1% | 2% | 3% | 2% | | 40% |
| 6 | 47% | 51% | 2% | 1% | 2% | 1% | | 40% |
| 7 | 50% | 50% | | 5% | 0.5% | 15% | | 30% |
| 8 | 50% | 50% | | 8% | 0.8% | 20% | | 30% |
| 9 | 50% | 50% | 3% | | 1% | 25% | | 30% |
| 10 | 38.5% | 61.5% | | 1.5% | 5% | 5% | | 35% |
| 11 | 52% | 48% | | | 7% | 4% | | 35% |
| 12 | 33% | 60% | 5% | 2% | 6% | 3% | | 35% |
| 13 | 30% | 70% | | | 8% | 2% | | 45% |
| 14 | 50% | 45% | | | 4% | 5% | 2% | 40% |
| | wheat | guar gum | | | sunflower | | carnauba | |
| | starch | 5% xanthan | | | oil | | wax | |
| | | gum | | | | | | |
| 15 | 55% | 40% | | 1% | 3% | 5% | | 40% |
| | corn | guar gum | | carnauba | sunflower | | | |
| | starch | 5% xanthan | | wax | oil | | | |
| | | gum | | | | | | |
| 16 | 52% | 43% | | | 4% | 5% | 2% | 40% |
| | corn | guar gum | | | sunflower | | carnauba | |
| | starch | 5% xanthan | | | oil | | wax | |
| | | gum | | | | | | |
| 17 | 50% | 45% | | | 2% | | 4% | 30% |
| | Tapioca- | guar gum | | | nut oil | | canola | |
| | starch | 5% xanthan | | | | | wax | |
| | | gum | | | | | | |
| 18 | 55% | 40% | | 2% | 4% | 10% | | 40% |
| | wheat | guar gum | | soy wax | sunflower | | | |
| | starch | 5% xanthan | | | oil | | | |
| | | gum | | | | | | |
| 19 | 65% | 30% | | | 4% | | | 35% |
| | | guar gum | | | | | | |
| | | 5% xanthan | | | | | | |
| | | gum | | | | | | |
| 20 | 50% | 45% | | | 4% | | | 40% |
| | | guar gum | | | | | | |
| | | 5% xanthan | | | | | | |
| | | gum | | | | | | |
| 21 | 50% | 45% | | | 4% | | 2% | 40% |
| | wheat | guar gum | | | sunflower | | carnauba | |
| | starch | 5% xanthan | | | oil | | wax | |
| | | gum | | | | | | |
| Min. | 30%-70% | 30%-70% | 0%-10% | 0%-10% | 0.5%-10% | 1%-25% | 0%-10% | 30-50% |
| Max. | | | | | | | | |

Step b:

Forming the mixture from step a. into an aid for eating or drinking or into a medical supply.

In step b., the mixture from step a. is formed into an aid such as a drinking straw. Step b. is explained below using the drinking straw as an example. However, any shape can be produced by changing the casting die.

The mixture from step a is delivered into a funnel via an outlet valve from the mixing device. The mixture is transported further via a “Screw Conveyor”. In a “Single Screw Extruder” machine, the mixture is transported to an outlet by means of a screw (plasticizing screw) and pressed into its shape through an outlet. This forms the desired drinking straw shape. The plasticizing screw tapers towards the end at which the outlet is located, i.e., the diameter of the screw decreases from the inlet to the outlet, so that the pressure on the mixture passing through the screw increases as it approaches the outlet. The plasticizing screw itself is arranged in a tube which is surrounded by various heating bands. A water-cooling system is can also be arranged between the heating bands and the tube. This ensures that the screw does not overheat. The heating bands can have different temperatures. Heating bands are arranged in succession from the inlet to the outlet of the screw in the tube. The temperatures of these heating bands can be 50° C., 65° C., 70° C. and 50° C. Such an arrangement is intended to ensure that the temperature of the material is constantly maintained between 50° C. and 70° C. The temperature of the mass at this step is decisive for the strength and pliability of the material. A temperature that is too low does not activate the gelation reaction, which can make the material of the drinking straw brittle. Too high a temperature, however, will cause the material to show signs of burning such as discoloration, become brittle and/or fragile. Too high a temperature can also cause bubbles to form in the material. Both temperature extremes are thus undesirable.

The narrower shape of the plasticizing screw, tapering towards the outlet, results in ever-increasing pressure on the compound moving through the screw, which can also lead to an increase in temperature. The last heating band has a lower temperature because the heat in the compound is generated by pressure and friction. Best results were obtained with a screw speed of 30 Hz to 50 Hz. This causes the material of the mixture from step a to be compressed very tightly during the forming of the disclosed aids, resulting in a high material density, which contributes to the breaking strength of the drinking straws. This material density makes the drinking straw very hard and unbreakable. Furthermore, the high pressure also forces out the water in the mixture, which is discharged through nozzles in the form of water vapor. The compacted material is pressed into a funnel-shaped collecting basin located at the outlet of the plasticizing screw. This initially fills up with the material. Subsequently, the pressure of the mass constantly flowing out of the outlet of the screw forces the mass in the collecting basin through another outlet in the collecting basin. The outlet comprises several round molds, such as dies, which lead to the shaping of several drinking straws (or other elongated aids such as a lollipop stick or a holding stick of an ear swab). The (drinking straw) strands thus formed by extrusion are placed on a comb and transported further by means of a conveyor belt. The formed strands are hot (approx. 66° C.) and the water remaining in the material evaporates. The strands for drinking straws have a wall thickness of 1 mm and a diameter of 7 mm.

The so-called “pressing process” is quite time-saving, such that step b can be carried out in a period of 4-10 minutes, preferably 5 minutes (from addition of the mixture to the pressed drinking straw strand leaving the outlet of the collecting basin).

The same procedure can be used to form a lollipop handle. The diameter of the outlets is then smaller. A lollipop handle/lollipop stick of the present invention can have a diameter of 3 mm, a wall thickness of 1 mm and a length of 12 cm.

The same process is used to form a holding stick for an ear swab.

Forming other aids for eating or drinking in addition to medical supplies:

To obtain, for example, cutlery or other “flat” aids or supplies, the mixture is extruded via the outlet after being compacted by means of a plasticizing screw. The outlet has the shape of an unclosed circle. As soon as the pressed mass rests on the surface of a conveyor belt, the two “ends of the circle” fold away to the side and a flat belt of the mass is formed. This is conveyed by conveyor belt to a rolling machine where it is rolled into a dough sheet of desired thickness. Then the desired aids for eating or drinking, such as cutlery like forks, spoons and knives, or medical supplies like tongue depressors, are cut out from the rolled dough sheet using a cylinder. The die used for cutting permits the shaping of the desired utensil by pressing the flat dough sheet into the desired two- or three-dimensional shape. This forms the desired cutlery shape.

Step c:

In step c., the aids formed in step b are hardened. This can be done, for example, by drying using aeration/ventilation. In this process, the shaped aids are introduced into a “cooling and cutter” machine (also called a cooling tunnel), where they are dried by fans and thus hardened. The subsequent curing by means of heat in a heating tunnel or drying tunnel is carried out at a temperature range between 25 and 100° C., preferably 85° C. In this process, air at the appropriate temperature is introduced through the tunnel.

The process for producing the aid may optionally comprise further steps. For example, if the aid is a drinking straw, lollipop handle/lollipop stick, holding stick for ear swabs or similar elongated aid, the respective strands formed in step b may be shortened in length to form individual drinking straws, lollipop handles/lollipop sticks, a holding stick for ear swabs or any similarly elongated aid. This is done in the “cooling and cutter” machine by, for example, a knife.

The process for producing the aids may also include irradiating the aids with UV light. This is used to disinfect the aids and supplies.

In some embodiments, the aid is further printed. Any suitable printing process known to the skilled person can be used for printing, for example flexographic printing processes or laser printing processes. For example, a printing machine from Guowei, GWR, or Hangzhou Colon Machinery Co., Ltd., model CL-DC850, may be used. Preferably, the printing is done with non-toxic inks. In particularly preferred embodiments, food coloring is used for printing.

In this case, steps b and c have a total duration of less than 30 min, preferably 20 minutes. The entire manufacturing process thus has a total duration of 25-35 min, preferably 25 minutes. This very short total duration is reflected in low energy consumption, which makes the process economical.

The processes of the present invention can produce a large quantity of aids for eating or drinking or medical supplies in a short time. For example, the output of the process for producing drinking straws may be at least 50 kg/h, preferably at least 100 kg/h, more preferably at least 150 kg/h.

The output of the process for producing cutlery can be, for example, at least 50 kg/h, preferably at least 100 kg/h, more preferably at least 150 kg/h.

Moreover, due to this short time, the energy to be spent for this purpose is very low in contrast to usual drying processes in e.g., a drying tunnel.

Step d (optional):

In order to increase the resistance to moisture such as water, the aids for eating or drinking and medical supplies produced by the present process can be coated with a thin coating based on wax or natural latex. Furthermore, the coating according to the invention imparts further advantageous properties to the drinking straw or the aids—the coating may produce less friction on the lips of the person consuming or drinking, and sticking of the drinking straw or the aids can thus also be avoided. Thus, sticking together of individual aids such as drinking straws or supplies such as cotton swabs during storage or transport is also prevented.

For this purpose, the wax and/or the natural latex can be present in an emulsion and is applied, e.g., sprayed, onto the drinking straws or cutlery. The preferred wax in this case is carnauba wax. A preferred wax emulsion consists of 30-50% wax, preferably carnauba wax or canola wax, 50-70% water and optionally excipients and/or one or more anti-stick additives. Preferably, the wax-based emulsion contains 32.5% carnauba wax, 17.5% kerosene and 50% water or consists of 32.5% carnauba wax, 17.5% natural latex and 50% water. Another preferred wax emulsion is 50% carnauba wax and 50% water. A natural latex-based emulsion consists of 50-95% natural latex 5-10% wax, preferably carnauba wax or canola wax, and optionally excipients and/or one or more anti-stick additives. Preferably, the natural latex-based emulsion contains 10% carnauba wax and 90% rubber emulsion consisting of 60% solid rubber and 40% water, or natural latex milk, or 5% carnauba wax, 45% rubber and 50% water, or of 5% canola wax, 45% rubber and 50% water, or of 5% carnauba wax and 95% natural latex, or of 5% carnauba wax, 2% glycerin, 43% natural latex and 50% water.

For all wax emulsions for coating, soy wax, beeswax or kerosene wax can be used alternatively. Further, the emulsions may also comprise one or more biopolymers and/or excipients.

Preferably, the emulsion for coating the excipients and supplies further comprises one or more anti-stick additives which significantly reduce friction and sticking between individual aids and supplies and thus prevent sticking to each other during storage in, for example, a smaller container. These anti-stick additives include oils such as canola oil, coconut oil, sunflower oil, lecithins such as sunflower lecithin or soy lecithin, and vegetable magnesium stearate. The one or more oils, preferably canola oil, can be added to the emulsion in an amount up to 5%, preferably 0.5%. If oil is added to the emulsion as an anti-stick additive, further addition of sunflower lecithin or soy lecithin is beneficial. It helps in blending the components of the emulsion. In addition, vegetable magnesium stearate may be added to the emulsion in an amount up to 3%, preferably 0.25-0.75%. The emulsion may further comprise glycerin.

To an emulsion comprising glycerin, the glycerin can either be added directly to the emulsion or sprayed as a second coating onto the first coating based on the emulsion. This serves as a lubricating film and prevents sticking.

Coating with the emulsion can be done in different ways. The coating may be present only on the outside or only on the inside surfaces or may be present on both the outside and the inside surfaces.

In one embodiment, the aids and supplies are briefly placed in a basin of wax emulsion and then dried.

In another embodiment, the aids and supplies are conveyed to a coating machine where they are coated with a wax emulsion using spray nozzles and then dried. When applied by spray, ethanol may be added to the emulsion, optionally in an amount up to 2%, preferably 0.3%.

An applied coating also provides the aid a smooth and glossy surface.

To reduce friction and adhesion of the individual aids or supplies during storage, the emulsion-coated aid or supply can be dusted with a thin layer of a powdered component such as stearate or flour as an alternative to admixing one or more anti-stick additives. For this purpose, the respective aid or supply, for example drinking straws, are placed in a so-called “dust box”, rotated therein, removed and then the excess powder is tapped off.

### Preferred Embodiments

In one preferred embodiment, the method for the production of aids for eating or drinking and for the production of medical supplies comprises the following steps:
a. Preparing a mixture comprising vegetable starch and vegetable thickening or gelling agent, comprising the steps of:
   a.1 combining the solid components of the mixture containing
      50% wheat starch,
      45% guar gum,
      5% xanthan gum,
      and mixing same,
   a.2 adding the liquid components of the mixture to the mixture of step a.1 comprising based on the net weight of the mixture of step a.1
      2% carnauba wax emulsion,
      4% sunflower oil,
      5% glycerin, and
      40% water,
      and mixing of same,
b. forming the mixture from step a. into an aid for eating or drinking or a medical supply,
c. curing the aid for eating or drinking or medical supply formed in step b,
d. optionally, coating the aid for eating or drinking or medical supply cured in step c. with a wax- or natural latex-based emulsion.

In another preferred embodiment, the method for the production of aids for eating or drinking and for making medical supplies comprises the following steps:
a. Preparing a mixture comprising vegetable starch and vegetable thickening or gelling agent, comprising the steps of:
   a.1 combining solid components of the mixture containing
      55% corn starch,
      40% guar gum,
      5% xanthan gum, and
      based on the net weight of said mixture,
      1% carnauba wax powder,
      and mixing of same,
   a.2 adding liquid components of the mixture to the mixture of step a.1 comprising based on the net weight of the mixture of step a.1
      3% sunflower oil,
      5% glycerin, and
      40% water,
      and mixing of same,
b. forming the mixture from step a. into an aid for eating or drinking or a medical supply,
c. curing the aid for eating or drinking or medical supply formed in step b,
d. optionally, coating the aid for eating or drinking or medical supply cured in step c. with a wax- or natural latex-based emulsion.

In a preferred embodiment, the method for the production of aids for eating or drinking and for the preparation of medical supplies comprises the following steps:
a. Preparing a mixture comprising vegetable starch and vegetable thickening or gelling agent, comprising the steps of:
   a.1 combining the solid components of the mixture containing
      52% corn starch,
      43% guar gum,
      5% xanthan gum,
      and mixing of same,
   a.2 adding the liquid components of the mixture to the mixture of step a.1 comprising based on the net weight of the mixture of step a.1
      2% carnauba wax emulsion,
      4% sunflower oil,
      5% glycerin, and
      40%, water
      and mixing of same,
b. forming the mixture from step a. into an aid for eating or drinking or a medical supply,
c. curing the aid for eating or drinking or medical supply formed in step b,
d. optionally, coating the aid for eating or drinking or medical supply cured in step c. with a wax- or natural latex-based emulsion.

In another preferred embodiment, the method for the production of aids for eating or drinking and for making medical supplies comprises the following steps:
a. preparing a mixture comprising vegetable starch and vegetable thickening or gelling agent, comprising the steps of
   a.1 combining solid components of the mixture containing
      50% tapioca starch,
      45% guar gum,
      5% xanthan gum,
      and mixing of same,
   a.2 adding liquid components of the mixture to the mixture of step a.1 comprising based on the net weight of the mixture from step a.1
      4% canola wax emulsion,
      2% nut oil,
      5% glycerin, and
      30% water,
      and mixing of same
b. forming the mixture from step a. into an aid for eating or drinking or a medical supply,
c. curing the aid for eating or drinking or medical supply formed in step b.,
d. optionally, coating the aid for eating or drinking or medical supply cured in step c. with a wax- or natural latex-based emulsion.

In yet another preferred embodiment, the method for the production of aids for eating or drinking and for the preparation of medical supplies comprises the following steps:
a. preparing a mixture comprising vegetable starch and vegetable thickening or gelling agent, comprising the steps of
   a.1 combining solid components of the mixture containing
      55% wheat starch,
      40% guar gum,
      5% xanthan gum, and
      based on the net weight of said mixture,
      2% soy wax powder,
      and mixing of same,
   a.2 adding liquid components of the mixture to the mixture of step a.1 comprising based on the net weight of the mixture of step a.1
      4% sunflower oil,
      10% glycerin, and
      40% water,
      and mixing of same,
b. forming the mixture from step a. into an aid for eating or drinking or a medical supply,
c. curing the aid for eating or drinking or medical supply formed in step b,
d. optionally, coating the aid for eating or drinking or medical supply cured in step c. with a wax- or natural latex-based emulsion.

In a preferred embodiment, the method for the production of aids for eating or drinking and for the preparation of medical supplies comprises the following steps:
a. preparing a mixture comprising vegetable starch and vegetable thickening or gelling agent, comprising the steps of:
   a.1 combining solid components of the mixture containing
      50% wheat starch,
      45% guar gum,
      5% xanthan gum,
      and mixing of same,
   a.2 adding liquid components of the mixture to the mixture of step a.1 comprising based on the net weight of the mixture of step a.1
      2% carnauba wax emulsion,
      4% sunflower oil,
      5% glycerin, and
      40% water,
      and mixing of same,
b. forming the mixture from step a. into an aid for eating or drinking or a medical supply,
c. curing the aid for eating or drinking or medical supply formed in step b,
d. optionally, coating the aid for eating or drinking or medical supply cured in step c. with a wax- or natural latex-based emulsion.

### Specific Embodiments

A method for the production of drinking straws comprising the following steps:
a. Preparing a mixture comprising vegetable starch and vegetable thickening or gelling agent, comprising the steps of:
   a.1 combining solid components of the mixture containing
      50% wheat starch,
      45% guar gum,
      5% xanthan gum,
      and mixing of same,
   a.2 adding liquid components of the mixture to the mixture of step a.1 comprising based on the net weight of the mixture from step a.1
      2% carnauba wax emulsion,
      4% sunflower oil,
      5% glycerin, and
      40% water,
      and mixing of same,
b. forming the mixture from step a. into a drinking straw,
c. curing the drinking straw formed in step b.,
d. coating the drinking straw cured in step c. with a natural latex emulsion,
   wherein the carnauba wax emulsion comprises 50% carnauba wax and 50% water, and
   wherein the wax emulsion for coating consists of 5% carnauba wax, 2% glycerin, 43% natural latex and 50% water and optionally contains one or more anti-stick additives.

A method for the manufacture of drinking straws comprises the following steps:
a. Preparing a mixture comprising vegetable starch and vegetable thickener or gelling agent, comprising the steps of:
   a.1 combining solid components of the mixture containing
      50% wheat starch,
      45% guar gum,
      5% xanthan gum,
   and mixing of same,
   a.2 adding liquid components of the mixture to the mixture of step a.1 comprising based on the net weight of the mixture from step a.1
      2% carnauba wax emulsion,
      4% sunflower oil,
      5% glycerin, and
      40% water,
      and mixing of same,
b. forming the mixture from step a. into a drinking straw,
c. curing the drinking straw formed in step b.,
d. coating the drinking straw cured in step c. with a natural latex emulsion,
   wherein the carnauba wax emulsion comprises 50% carnauba wax and 50% water, and
   wherein the wax emulsion for coating comprises 5% carnauba wax, 95% natural latex or rubber emulsion, and optionally one or more anti-stick additives.

A method for the manufacture of drinking straws comprises the following steps:
a. Preparing a mixture comprising vegetable starch and vegetable thickener or gelling agent, comprising the steps of:
   a.1 combining solid components of the mixture containing
      55% wheat starch,
      40% guar gum,
      5% xanthan gum,
      and mixing of same,
   a.2 adding liquid components of the mixture to the mixture of step a.1 comprising based on the net weight of the mixture from step a.1
      2% soy wax powder,
      4% sunflower oil,
      10% glycerin,
      2% food coloring, and
      40% water,
      and mixing of same,
b. shaping the mixture from step a. into a drinking straw,
c. curing the drinking straw formed in step b.,
d. coating the drinking straw cured in step c. with a wax emulsion,
   wherein the wax emulsion for coating consists of 32.5% carnauba wax, 17.5% kerosene and 50% water and optionally contains one or more anti-stick additives.

A method for the production of cutlery comprises the following steps:
a. Preparing a mixture comprising vegetable starch and vegetable thickener or gelling agent, comprising the steps of:
   a.1 combining solid components of the mixture containing
      50% wheat starch,
      45% guar gum,
      5% xanthan gum,
      and mixing of same,
   a.2 adding liquid components of the mixture to the mixture of step a.1 comprising based on the net weight of the mixture from step a.1
      2% carnauba wax emulsion,
      4% sunflower oil,
      5% glycerin, and
      40% water,
      and mixing of same,
b. forming the mixture from step a. into cutlery,
c. curing the cutlery formed in step b.,
d. optionally, coating the cutlery cured in step c. with a wax emulsion,
   wherein the carnauba wax emulsion comprises 50% carnauba wax and 50% water, and
   wherein the wax emulsion for coating consists of 50% carnauba wax and 50% water and optionally contains one or more anti-stick additives.

A method for the production of lollipop sticks/lollipop handles comprises the following steps:
a. Preparing a mixture comprising vegetable starch and vegetable thickener or gelling agent, comprising the steps of:
   a.1 combining solid components of the mixture containing
      50% wheat starch,
      45% guar gum,
      5% xanthan gum,
      and mixing of same,
   a.2 adding liquid components of the mixture to the mixture of step a.1 comprising based on the net weight of the mixture from step a.1
      2% carnauba wax emulsion,
      4% sunflower oil,
      5% glycerin, and
      40% water,
      and mixing of same,
b. forming the mixture from step a. into a lollipop handle/lollipop stick,
c. curing the lollipop handle/lollipop stick formed in step b.,
d. optionally, coating the lollipop handle/lollipop stick cured in step c. with a wax emulsion,
   wherein the carnauba wax emulsion comprises 50% carnauba wax and 50% water, and
   wherein the wax emulsion to be coated consists of 32% carnauba wax, 17.5% natural latex and 50% water and optionally contains one or more anti-stick additives.

A method for the production of ear swabs comprises the following steps:
a. Preparing a mixture comprising vegetable starch and vegetable thickener or gelling agent, comprising the steps of:
   a.1 combining solid components of the mixture containing
      50% wheat starch,
      45% guar gum,
      5% xanthan gum,
      and mixing of same,
   a.2 adding liquid components of the mixture to the mixture of step a.1 comprising based on the net weight of the mixture from step a.1
      2% carnauba wax emulsion,
      4% sunflower oil,
      5% glycerin, and
      40% water,
      and mixing of same,
b. forming the mixture from step a. into an ear swab,
c. curing the ear swab formed in step b.,
d. optionally, coating the ear swab cured in step c. with a wax emulsion,
   wherein the carnauba wax emulsion comprises 50% carnauba wax and 50% water, and
   wherein the wax emulsion for coating consists of 32% carnauba wax, 17.5% natural latex and 50% water and optionally contains one or more anti-stick additives.

A method for the production of popsicle sticks comprises the following steps:
a. Preparing a mixture comprising vegetable starch and vegetable thickener or gelling agent, comprising the steps of
   a.1 combining solid components of the mixture containing
      50% wheat starch,
      45% guar gum,
      5% xanthan gum,
      and mixing of same,
   a.2 adding liquid components of the mixture to the mixture of step a.1 comprising based on the net weight of the mixture from step a.1
      2% nut oil,
      4% canola wax emulsion,
      30% water,
      and mixing of same,
b. forming the mixture from step a. into a popsicle stick,
c. curing the popsicle stick formed in step b.,
d. coating the popsicle stick cured in step c. with a wax emulsion,
   wherein the canola wax emulsion comprises 50% canola wax and 50% water, and
   wherein the wax emulsion for coating comprises 50% soy wax and 50% water and optionally contains one or more anti-stick additives.

### EXAMPLES

The present invention is illustrated by the following non-limiting examples.

### Example 1: Production of a Drinking Straw

The starting material for the drinking straw consists of 50% wheat starch, 45% guar gum, 5% xanthan gum. In addition, based on net weight plus 2% carnauba wax emulsion, 4% sunflower oil, 5% glycerin and 40% water. These raw materials, exclusively water, glycerin, carnauba wax emulsion and sunflower oil, were mixed in a blender. This is done until the various components are well mixed. Water (+40% on net weight), carnauba wax emulsion (+2% on net weight), glycerin (+5% on net weight) and sunflower oil (+4% on net weight) were then slowly added. The highly powdery composition has a temperature of about 35° C. This mixture was then transferred to a funnel via an outlet valve. The still very powdery mixture was then added to a “Screw Conveyor”, which transported the mixture in an upward direction. The mixture was then conveyed into the feeding system of the “Single Screw Extruder” machine. The mixture was heated to 60° C., 65° C., 65° C., and 50° C. by means of 4 successive heating bands. At the same time, the screw and cylinder were cooled by water cooling. The mixture was conveyed by a screw to the front to a collecting basin and then pressed through 10 outlets (dies) to form strands. This formed the desired preliminary drinking straw shape. The formed drinking straw strands had a wall thickness of 1 mm and a diameter of 7 mm. The strands were then processed in a “cooling and cutter” machine. The strands dried as they passed through the conveyor belt, where fans mounted on the top of the machine cooled down the still moist (35%) and warm (65° C.) material. After passing through the 10 m long cooling tunnel, the strands entered the cutting machine, which cut the strands to the desired length of 20 cm with a blade thrusting from top to bottom. Finally, the cut straws were conveyed on to the conveyor belt of a heating tunnel, which completely cured and disinfected the straws at approximately 85° C. hot air and UV light. At the end of the heating tunnel, the drinking straws were conveyed into a “coating machine”. This conveyed the drinking straws by means of a conveyor belt with slats into a basin with an emulsion consisting of: 5% carnauba wax, 2% glycerin, 43% natural latex and 50% water. Excipients and one or more anti-stick additives were also added. Thus, the drinking straws were coated on the inside as well as on the outside surfaces. Subsequently, the drinking straws were transported to another drying tunnel and the coating was further cured. Finally, the drinking straws were packaged into a cardboard box according to governing hygiene regulations.

### Example 2: Production of Cutlery (Knives)

The starting material for the cutlery (knives) consists of 55% corn starch, 40% guar gum, 5% xanthan gum. In addition, based on net weight plus 4% canola wax powder, 4% nut oil, 35% water and 4% blue food coloring. These raw materials, exclusively water, food coloring and nut oil, were mixed in a blender. This is done until the various components are thoroughly mixed. Water (+40% to net weight), food coloring (+4% to net weight) and nut oil (+4% to net weight) were then slowly added. The highly powdery composition has a temperature of about 35° C. This mixture was then poured into a funnel through an outlet valve. The still powdery mixture was then added to a “Screw Conveyor”, which transported the mixture in an upward direction. The mixture was then conveyed into the feeding system of the “Single Screw Extruder” machine. This was heated to 60° C., 65° C., 65° C., and 50° C. by means of 4 successive heating bands. At the same time, the screw and cylinder were cooled by water cooling. The mixture was conveyed here by a screw to the front to a collecting basin and then pressed through an outlet (die) into its shape of an almost closing circle. The mass opens and folds out. This formed the desired preliminary shape. With a wall thickness of 2 mm. The approx. 50 cm wide dough sheet is conveyed to the punching out area. The mass is then cut out of the flat (2 mm) dough plate using a rolling machine to achieve the desired shape (knife). This formed the desired cutlery shape, in this case a knife. The shaped knife has a thickness of 2 mm. Finally, the cut cutlery (knife) was conveyed onto the conveyor belt of a heating tunnel, which completely cured and disinfected the cutlery (knife) at approximately 85° C. hot air and UV light. At the end of the heating tunnel, the knives were conveyed to a coating machine. This coated the knives by means of spray nozzles with a wax emulsion consisting of 50% carnauba wax and 50% water. Excipients were also applied. The knives rotated once from side to side through the self-rotating cylinders, which simultaneously transported the knives forward through the rotation and dried them through fans mounted above them. Finally, the drinking straws were packaged into a cardboard box according to governing hygiene regulations.

### Example 3: Production of a Lollipop Handle/Lollipop Stick

The starting material for the lollipop handle/lollipop stick consisted of 52% corn starch, 43% guar gum, 5% xanthan gum. In addition, based on net weight, 1% carnauba wax powder, 3% sunflower oil, 2% food coloring and 40% water were added. These raw materials, excluding water, food coloring and sunflower oil, were blended in a blender until the various components are thoroughly mixed. Water (+40% to net weight), food coloring (+2% to net weight) and sunflower oil (+3% to net weight) were then slowly added. The highly powdery composition has a temperature of about 35° C. This mixture was then transferred to a funnel via an outlet valve. The powdery mixture was then added to a “Screw Conveyor”, which transported the mixture in an upward direction. The mixture was then conveyed into the feeding system of the “Single Screw Extruder” machine and heated to 60° C., 65° C., 65° C., and 50° C. by means of 4 successive heating bands. At the same time, the screw and cylinder were cooled by water cooling. The mixture was then conveyed by a screw to the front to a collecting basin and then pressed through 40 outlets (dies) into its shape to form strands. This formed the desired preliminary lollipop stick shape. The formed lollipop sticks had a wall thickness of 1 mm and a diameter of 3 mm. The lollipop strands were then processed in a “cooling and cutter” machine. The lollipop strands dried as they passed through the conveyor belt, where fans mounted on the top of the machine cooled down the still moist (35%) and warm (65C.°) material. After passing through the 10 m long cooling tunnel, the strands entered the cutting machine, which cut the strands to the desired length of 20 cm with a blade thrusting from top to bottom. Finally, the cut lollipop sticks were transferred to the conveyor belt of a heating tunnel, which completely cured and disinfected the lollipop sticks at approximately 90° C. hot air and UV light. At the end of the heating tunnel, the lollipop sticks were conveyed to a coating machine. This coated the lollipop sticks with an emulsion (32.5% carnauba wax, 17.5% natural latex and 50% water) using spray nozzles. Excipients and one or more anti-stick additives were also added. The lollipop handle/lollipop stick were rotated once 360 degrees through the self-rotating cylinders, which at the same time moved the lollipop handle/lollipop stick in a forward direction. Subsequently, the lollipop handle/lollipop stick was sprayed with a glycerin film. The lollipop handle/lollipop stick was rotated 360° through the self-rotating cylinders. Finally, the drinking straws were packaged into a cardboard box according to governing hygiene regulations.

### Example 4: Production of a Popsicle Stick

The starting material for the popsicle stick consists of 50% tapioca starch, 45% guar gum, 5% xanthan gum. In addition, based on net weight plus 2% nut oil, 4% canols wax emulsion and 30% water. These raw materials, exclusively water, canola wax emulsion and oil, were mixed in a blender. This is done until the various components are thoroughly mixed. Water (+40% to net weight), canola wax emulsion (+4% to net weight) and nut oil (+2% to net weight) were then slowly added. The highly powdery composition has a temperature of about 35° C. This mixture was then transferred to a funnel via an outlet valve. The still powdery mixture was then added to a “Screw Conveyor”, which transported the mixture in an upward direction. The mixture was then conveyed into the feeding system of the “Single Screw Extruder” machine. This was heated to 60° C., 65° C., 65° C., and 50° C. by means of 4 successive heating bands. At the same time, the screw and cylinder were cooled by water cooling. The mixture was conveyed here by a screw to the front to a collecting basin and then pressed through an outlet (die) into its shape of an almost closing circle. The mass opens and folds out. This formed the desired preliminary shape having a wall thickness of 2 mm. The 50 cm wide dough sheet is conveyed to the punching area, where the mass is punched out with a rolling machine to the desired popsicle stick from the flat (2 mm) dough sheet. This formed the desired popsicle stick shape. The shaped popsicle stick has a thickness of 2 mm. Finally, the cut popsicle stick was transferred to the conveyor belt of a heating tunnel, which completely cured and disinfected the popsicle stick at approximately 80° C. hot air and UV light. At the end of the heating tunnel, the popsicle sticks were conveyed to a coating machine, which coated the popsicle sticks with a wax emulsion (50% soy wax and 50% water) by means of spray nozzles. The popsicle sticks rotated from side to side through the self-rotating cylinders, which at the same time transported the popsicle sticks forward through the rotation and dried them through fans mounted above them. Finally, the drinking straws were packaged into a cardboard box according to governing hygiene regulations.

### Example 5: Production of a Drinking Straw

The starting material for the drinking straw consisted of 55% wheat starch, 40% guar gum, 5% xanthan gum. In addition, based on net weight, 2% soy wax powder, 4% sunflower oil, 10% glycerin, 2% food coloring and 40% water were added. These raw materials, excluding water, food coloring, glycerin and sunflower oil, were blended in a blender. This is done until the various components are thoroughly mixed. Water (+40% to net weight), glycerin (+10 to net weight), food color (+2% to net weight) and sunflower oil (+4% to net weight) were then slowly added. The highly powdery composition has a temperature of about 35° C. This mixture was then poured into a funnel via an outlet valve. The still very powdery mixture was then put into a “Screw Conveyor”, which transported the mixture in an upward direction. The mixture was then conveyed into the feeding system of the “Single Screw Extruder” machine. This was heated to 60° C., 65° C., 65° C., and 50° C. by means of 4 successive heating bands. At the same time, the screw and cylinder were cooled by water cooling. The mixture was then conveyed by a screw to the front to a collecting basin and then pressed through 10 outlets (dies) into its shape to form strands. This formed the desired preliminary drinking straw shape. The formed drinking straw strands had a wall thickness of 1 mm and a diameter of 5 mm. The strands were then processed in a “cooling and cutter” machine. In this process, the strands dried as they passed through the conveyor belt, where fans mounted on the top of the machine cooled the still moist (35%) and warm (65° C.) material. After passing through the 10 m long cooling tunnel, the strands entered the cutting machine, which cut the strands to the desired length of 18 cm with a top-down blade. Finally, the cut straws were transferred to the conveyor belt of a heating tunnel, which completely cured and disinfected the straws at approximately 85° C. hot air and UV light. At the end of the heating tunnel, the drinking straws were conveyed into a “coating machine”. This conveyed the drinking straws by means of a conveyor belt with slats into a basin with an emulsion consisting of 5% canola wax, 75% natural latex and 20% water. Furthermore, excipients and one or more anti-stick additives were added. Thus, the drinking straws were coated on the inside as well as on the outside. The straws were then transported to another drying tunnel where the coating was cured. The drinking straws were then rotated 360 degrees by the self-rotating cylinders, which simultaneously conveyed the drinking straws forward and sprayed them with a very thin layer of glycerine by means of spray nozzles. Finally, the drinking straws were packaged into a cardboard box according to governing hygiene regulations.

### Example 6: Coating of a Drinking Straw

The drinking straws were conveyed by means of a conveyor belt with slats into a basin containing a wax emulsion consisting of 32.5% carnauba wax, 17.5% kerosene and 50% water. Thus, the drinking straws were coated on the inside as well as on the outside surfaces. The drinking straws were then conveyed to another drying tunnel where the coating was cured.

Alternatively, the drinking straws are conveyed by means of a conveyor belt with slats into a basin with an emulsion consisting of 5% carnauba wax and 95% rubber emulsion or natural latex milk and additionally one or more anti-stick additives. Thus, the drinking straws were coated on the inside as well as on the outside. The drinking straws were then conveyed to another drying tunnel, where the coating cured. The drinking straws then rotated once through 360 degrees through the self-rotating cylinders, which simultaneously transported the drinking straws forward and sprayed them with a very thin layer of glycerine using spray nozzles. Finally, the drinking straws were packaged into a cardboard box according to governing hygiene regulations.

Alternatively, the drinking straws are conveyed by means of a conveyor belt with slats into a basin containing an emulsion consisting of 3% canola wax, 1% glycerin, 1% magnesium stearate and 95% rubber emulsion or natural latex milk, plus one or more anti-stick additives. Thus, the drinking straws were coated on the inside as well as on the outside surfaces. The drinking straws were then conveyed to another drying tunnel, where the coating cured. The drinking straws then rotated once through 360 degrees through the self-rotating cylinders, which simultaneously conveyed the drinking straws forward through the rotation and into a “dust box” and laid a dust film of magnesium stearate on the coating. Finally, the drinking straws were packaged into a cardboard box according to governing hygiene regulations.

### Example 7: Compostability of the Drinking Straws According to the Invention

The drinking straws according to the invention were tested for their compostability using two different procedures. In the first procedure, the test material was tested by “intensive rotting.” For this purpose, the material was placed in a rotting tunnel. After two turnover processes, the material was discharged and sieved. The test bag was checked prior to sieving. There was no more test material in the bag. The test material was therefore completely decomposed after 4 weeks, and thus composted.

In a second procedure, the test material was tested by dry fermentation. The test material was placed in a digester tunnel. After 27 days, the degree of composting was tested. The material was completely dissolved, and thus composted after 27 days.

1. A composition for forming an aid for eating or drinking or a medical supply, comprising:
vegetable starch and
a vegetable thickening or gelling agent,
wherein the aid or the medical supply is compostable, and
wherein the aid or the medical supply further comprises:
40-50% vegetable starch,
35-50% thickening or gelling agent,
0-10% cellulose and/or wood pulp,
0-10% wax,
0-3% nut oil or sunflower oil, and
0-2% glycerin.

2. (canceled)

3. (canceled)

4. (canceled)

5. The composition according to claim 1, further comprising wax wherein the wax is carnauba wax or soy wax.

6. (canceled)

7. The composition according to claim 1, wherein the aid or the medical supply further comprises an outer coating.

8. The composition according to claim 7, wherein the outer coating comprises carnauba wax, and/or canola wax, and/or paraffin, or mixtures thereof.

9. The composition according to claim 1, wherein the vegetable starch is selected from wheat starch, potato starch, corn starch, tapioca starch or a mixture thereof, and wherein the vegetable thickening or gelling agent is a mixture of guar gum and xanthan gum.

10. (canceled)

11. The composition of claim 1, wherein glycerin is sprayed on the aid or the medical supply as a second coating.

12. (canceled)

13. The composition according to claim 1, wherein the aid is a drinking straw, a cup, cutlery or a lollipop stick.

14. The composition according to claim 1, wherein the aid for eating or drinking comprises:
40-50% starch,
35-45% guar gum,
1-5% xanthan gum,
colorant, and a
natural latex-based coating,
wherein the coating consists of 5% carnauba wax, 2% glycerin, 93% natural latex.

15. (canceled)

16. (canceled)

17. (canceled)

18. (canceled)

19. (canceled)

20. (canceled)

21. (canceled)

22. The composition according to claim 7, wherein the outer coating comprises a biopolymers.

23. (canceled)

24. The composition according to claim 1, wherein the medical supply comprises:
40-50% vegetable starch,
35-45% guar gum,
1-5% xanthan gum, and optionally
a wax-based coating.

25. The composition according to claim 1, wherein the medical supply is a cotton swab, a tongue depressor, or a disposable toothbrush.

26. A method for the production of a compostable aid for eating or drinking or fora compostable medical supply formed by the composition according to claim 1, the method comprising the following steps:
(a). preparing a mixture comprising vegetable starch and a vegetable thickening or gelling agent comprising the steps of:
(i) combining solid ingredients of the mixture comprising vegetable starch and vegetable thickening or gelling agent and mixing same,
(ii) adding liquid ingredients comprising water, in an amount of 30-60% of the net weight of the mixture of (a)(i) to the mixture of step (a) (i) and mixing of same,
(b). forming the mixture of step (a) into an aid for eating or drinking or into the medical supply, and
(c). curing the aid for eating or drinking or the medical supply formed in step (b).

27. (canceled)

28. The method according to claim 26, wherein the vegetable starch in the mixture of step (a)(i) is wheat starch, potato starch, corn starch, tapioca starch or a mixture thereof and wherein the vegetable thickening or gelling agent in the mixture of step (a)(i) is a mixture of guar gum and xanthan gum.

29. The method according to claim 26, wherein the solid ingredients in step (a)(i) further comprise cellulose and/or wood pulp and/or wax powder.

30. (canceled)

31. (canceled)

32. The method according to claim 35, wherein the emulsion for the coating of step (d) consists either of 32.5% carnauba wax, 17.5% natural latex and 50% water, or of 50% carnauba wax and 50% water, or of 5% carnauba wax and 95% natural latex.

33. The composition according to claim 22, wherein the biopolymer is a natural latex excipient and/or an anti-stick additive.

34. The composition according to claim 14, wherein the aid is a compostable drinking straw.

35. The method according to claim 26, the method further comprising:
(d) coating the aid for eating or drinking or the medical supply cured in step (c) with an emulsion comprising carnauba wax, and/or canola wax, and/or paraffin, or mixtures thereof, preferably further comprising a biopolymer.

36. The method according to claim 35, wherein the emulsion comprises a biopolymer.

37. The method according to claim 36, wherein the biopolymer is natural latex.
